(19) United States
(12) Patent Application Publication
(10) Pub. No.: US 2022/0409753 A1
(43) Pub. Date: Dec. 29, 2022

(54) PHOTOSENSITISING COMPOSITION AND USES THEREOF

(71) Applicants: DENTSPLY SIRONA INC., York, PA (US); DENTSPY SIRONA INC., York, PA (US)

(72) Inventors: Anil KISHEN, Singapore (SG); Saji GEORGE, Singapore (SG); Chiew Lian Jennifer NEO, Singapore (SG); Keang-Peng SONG, Petaling Jaya (MY)

(73) Assignee: DENTSPLY SIRONA INC., York, PA (US)

(21) Appl. No.: 17/894,265

(22) Filed: Aug. 24, 2022

Related U.S. Application Data
(63) Continuation of application No. 17/017,881, filed on Sep. 11, 2020, now abandoned.
Application No. 17/017,881 is a continuation of application No. 11/922,044, filed on Jul. 16, 2008, now abandoned.
(60) Provisional application No. 60/689,997, filed on Jun. 13, 2005.

(51) Int. Cl. A61L 2/00; A61Q 11/00; A61K 31/045; A61K 8/34; A61K 41/00; A61K 6/52; A61K 6/56

(52) U.S. Cl. CPC A61L 2/0011; A61Q 11/00; A61K 31/045; A61K 8/34; A61K 8/345; A61K 41/0057; A61K 41/0071; A61K 6/52; A61K 6/56; A61K 2800/434

(57) ABSTRACT

The present invention provides a photosensitising composition comprising a mixture of: at least one surfactant; at least one alcohol; and/or water, with the proviso that when the at least one surfactant is an alcohol, it is different from the at least one alcohol. In particular, the composition may comprise a mixture of glycerol, ethanol and water. Alternatively, the composition may comprise a mixture of polyethylene glycol, ethanol and water. The photosensitising composition may further comprise at least one photosensitising compound. The present invention also provides a method of treating and/or preventing conditions caused by microorganisms in a subject.

### FIELD OF THE INVENTION

The present invention relates to a photosensitising composition, and its uses thereof.

### BACKGROUND OF THE INVENTION

Apical periodontitis, is defined as an inflammatory process around the tooth root-apex, and is primarily a sequel to microbial infection (mainly bacteria) of the root-canal space of the tooth. Infection of the root canal and associated regions of tooth, generally known as root canal infection/endodontic infection, is a widespread problem all over the world (Figdor D, 2002). It represents a localized infection where bacteria have been recognized as the main etiological agent (Tronstad L and Sunde T P, 2003). The clinical manifestation of the disease is due to the combined action of microorganisms and host immune response (Haapasalo M et al, 2003; Nair P N R, 2004).

The main objective of the clinical management of apical periodontitis (root canal infection) is to eliminate bacteria from the root canal system, and this is the only viable alternative to tooth loss. Root canal treatment (RCT) aims to disinfect the root canal by removing the infected tissue by means of ‘chemo-mechanical’ preparation. The complete disinfection of the root canal is not achieved in all the cases, although in most cases the disease symptoms recede. It is estimated from census data that in the year 1990, an estimated 14 million root canal treatments were performed in the USA alone. Interestingly, epidemiological studies of root-filled teeth in various countries and different populations have demonstrated the presence of apical periodontitis in a relatively high proportion (De Moor R J et al, 2000; Eckerbom M et al, 1989; Lupi-Pegurier L et al, 2002; and Dugas N N et al., 2003). In a large study in a Belgian population from 4617 teeth, apical periodontitis was found in 40.4% of all Root Canal Treated (RCT) teeth. In another study in Denmark, the periapical health of nearly 600 RCT teeth was compared and apical periodontitis was observed in approximately 50% of the RCT teeth. In two selected Canadian populations; the prevalence of apical periodontitis in RCT teeth was 44% and 51%. It should be emphasized that the great majority of RCT teeth with epical periodontitis are symptom free (Figdor D, 1996), and hence has not attracted the attention it deserves.

The success rate of root canal treatment has generally been regarded as high, of the order of 87% (Eriksen H M, 1998). This figure applies to root canal treatment carried out by a specialist, where a higher expertise would result in a better technical standard of treatment, whereas the success rate in general practice is of the order of 72%. Moreover, when the failure rate is measured relative to the prevalence of root canal treatment, the full dimension of the problem becomes apparent. It is estimated that there are 3.3 million failed root canal treatments in Australia and 54 million in the USA; taking the greater failure rate for treatment in general practice. Figdor D, 2002, suggested that if these numbers are multiplied by the cost of RC re-treatment and restoration replacement, the cumulative economic impact is in the order of billions of dollars (Figdor, 1996). Mostly failure of conventional treatment is due to the persistence of bacterial population even after chemo-mechanical disinfection. Limitations in conventional treatment procedures are attributed to its inability to reach bacterial biofilm, especially in anatomically inaccessible regions of tooth. The presence of biofilms, which is the surface adsorbed growth of microorganism, has been associated with chronic human infections (Costerton J W et al, 1994; Parsek M R and Singh P K, 2003). This is because bacteria growing biofilms are highly resistant to conventionally used antimicrobial regimes, due to the biochemical composition of biofilm matrix and altered physiology of bacteria residing in biofilms (Parsek M R and Singh P K, 2003).

Traditionally, an instrumentation procedure (with root canal reamers and files) supplemented with root canal irrigants (liquid chemicals) and medicaments are used to achieve a ‘bacteria free’ root canal system in RCT. The chemicals most commonly employed are sodium hypochlorite, chlorhexidine and EDTA, while calcium hydroxide is also used as an effective intra-canal medicament. These chemicals have to be supplemented with mechanical instrumentation to achieve bacterial elimination within the root canals.

Further, this method of bacterial elimination is not an instantaneous process and is found to be least effective in the anatomical complexities of the root canals. In the past, efforts were made to use higher concentration of chemicals to achieve effective bacterial elimination. However, some of the perennial concerns were not examined. The effectiveness of these chemicals (such as root canal irrigants) at various depths inside the dentinal tubule is not clear. It is demonstrated that the effective penetration of these chemicals into the dentinal tubules is limited, and therefore, bacteria remained viable at greater depths in the dentinal tubules at all levels in the root canal. Also, long term use of such chemicals and medicaments can lead to the development of resistance to the chemicals and medicaments in the target organisms. Besides, studies have shown that sodium hypochlorite reduces the modulus of elasticity and flexure strength of dentine structure, while saturated calcium hydroxide reduces the flexure strength of dentine. It is also observed that some of the common root canal pathogens such as Enterococcus faecalis and Candida albicans are resistant to calcium hydroxide.

Persistence of bacteria within the root canal dentine after RCT is usually the main cause of failure in root canal treatment. Recently, photodynamic therapy (PDT) has emerged as a promising treatment of cancer and other diseases utilizing activation of an external chemical agent, called photosensitiser or PDT drug, by light. This drug is administered either intravenously or topically to the malignant site, as in the case of certain skin cancers. Subsequently, light of a specific wavelength, which can be absorbed by the PDT photosensitiser, is applied. The PDT drug absorbs this light producing reactive oxygen species that can destroy the tumour. The photosensitizing compound is activated at a specific wavelength of light to destroy the target cell via a strong oxidizer, which causes cellular damage, membrane lysis and protein inactivation.

PDT relies on the greater affinity of the PDT drug for malignant cells. The light activation process of a PDT drug is initiated by the absorption of light to produce an excited singlet state (S1 or often written as 1P*, where P* represents the excited photosensitiser) which then populates a long-lived triplet state T1 (or 3P*) by intersystem crossing. It is the long-lived triplet state that predominantly generates the reactive oxygen species. Two types of processes have been proposed to produce reactive species that oxidize the cellular components (hence produce photooxidation) (Ochsner M, 1997).

Recent studies have shown that it is possible to kill bacteria, virus and fungi with low-power light/laser using the principles of photodynamic therapy (PDT) (Hamblin M R and Hasan T, 2004; O'Neill J F et al, 2002; Wainwright M, 1998; Jori G and Brown S B, 2004). PDT does not use photothermal effect such as high powered lasers to eradicate bacteria. Therefore, they circumvent issues of thermal side effects in tissues. PDT has been used with relative success in the field of oncology for the treatment of neoplastic cells.

Different photosensitisers have been successfully demonstrated to have antibacterial property with their potential use in treating localized infections (Wainwright, M, 1998). Since the bactericidal activity of PDT is based on oxygen free radicals, the chance of microbes developing resistance is minimum compared to other strategies (Hamblin M R and Hasan T, 2004; Wainwright M and Crossley K B, 2004). Different photosensitisers have been successfully demonstrated to have antibacterial property with their potential use in treating localized infections (Wainwright M, 1998).

Since free radical generation is highly dependent on environmental conditions, the physicochemical environment existing at the site of application can influence the outcome of the treatment. There is therefore a need in the state of the art to provide a more suitable photosensitising composition to increase the effectiveness of PDT.

There is also a need in the state of the art for treatment of conditions caused by microorganisms which are non-invasive, do not compromise on the health and integrity of non-infected tissue and/or structure, reduce or eliminate the use of antibiotics which may lead to gastrointestinal problems and bacterial resistance, and reduce labour and costs.

### SUMMARY OF THE INVENTION

The present invention seeks to address the problems above, and in particular provides a new photosensitising composition and its uses thereof. In particular, there is provided a photosensitising composition comprising a mixture of: at least one surfactant, at least one alcohol, and optionally water. The composition may further comprise at least one photosensitising compound.

According to a first aspect, the present invention provides a photosensitising composition comprising a mixture of: at least one surfactant; at least one alcohol; and/or water, wherein the ratio of the volume of the at least one surfactant to the at least one alcohol to water may be in the range of 10:5:85 to 40:30:30, with the proviso that when the at least one surfactant is an alcohol, it is different from the at least one alcohol. In particular, the ratio may be in the range of 15:10:75 to 35:25:40. Even more in particular, the ratio is 30:20:50. The at least one surfactant may be a trihydric alcohol or a polyester. The trihydric alcohol may be glycerol. The polyester may be polyethylene glycol or polypropylene glycol. The at least one surfactant may be selected from the group consisting of: mineral oil, glycerol, polyethylene glycol, Triton X, polypropylene glycol, SDS and any detergent suitable for use for the present invention. The alcohol may be a monohydric alcohol. In particular, the alcohol may be ethanol.

According to a particular aspect, the present invention provides a composition comprising a mixture of polyethylene glycol, ethanol and water. According to another particular aspect, the present invention also provides a composition comprising a mixture of glycerol, ethanol and water. The ratio of the volume of glycerol to ethanol to water may be 30:20:50.

The composition according to any aspect of the present invention may further comprise at least one photosensitising compound. The at least one photosensitising compound may be selected from the group consisting of: toluidene blue, methylene blue, arianor steel blue, tryptan blue, crystal violet, azure blue cert, azure B chloride, azure 2, azure A chloride, azure B tetrafluoroborate, thionin, azure A eosinate, azure B eosinate, azure mix sicc, azure II eosinate, haematoporphyrin HCl, haematoporphyrin ester, aluminium disulphonated phthalocyanine, chlorins, photoactive fullerenes (e.g. C16-b), aminolevulinic acid (ALA), bacteriochlorins, phthalocyanines, pheophorbides, purpurins, naphthalocyanines, indocyanine green, or mixtures thereof. In particular, the at least one photosensitising compound is methylene blue.

According to a particular aspect of the present invention, it is provided a composition comprising at least one photosensitising compound and a mixture of glycerol, ethanol and water. According to another particular aspect, the present invention provides a composition comprising at least one photosensitising compound and a mixture of polyethylene glycol, ethanol and water.

The composition may further comprise at least one of the following:
(a) an oxygen carrier;
(b) a polycationic compound; or
(c) an oxidising agent,
   or mixtures thereof.

The oxygen carrier may be selected from the group consisting of but not limited to: hydrofluoro carbons, perfluoro carbons or a mixture thereof. For example, the oxygen carrier may be selected from the group consisting of, but not limited to, perfluorodecahydro naphthalene, perfluorodecalin, perfluorohexane, octafluoropropane, perfluorobutane, perfluorooctane and perfluoromethyldecalin. The polycationic compound may be selected from the group consisting of, but not limited to: cationic polypeptides, such as poly L-lysine, L-arginine, D-arginine, and multivalent cations, such as calcium chloride, calcium hydroxide, magnesium chloride. The oxidising agents may be selected from the group consisting of, but not limited to: dilute sodium hypochlorite, hydrogen peroxide, DMSO and chlorine dioxide.

The composition may further comprise a pharmaceutically acceptable excipient and/or carrier.

According to a particular aspect, the composition may be formulated for use in oral cavity treatment. The composition may be formulated for use in the treatment and/or prevention of conditions caused by microorganisms. The composition may be formulated for use in the treatment and/or prevention of periodontal and/or halitosis conditions. The composition may also be formulated for topical administration or administration by injection.

According to a further aspect, the composition may be for use in medicine.

The present invention also provides a use of a photosensitising composition according to any aspect of the present invention in the manufacture of a medicament for treating and/or preventing conditions caused by microorganisms in a subject, the treatment and/or prevention comprising the steps of:
(a) administering the photosensitising composition; and
(b) irradiating the area to which the composition is administered with light at a wavelength absorbed by a photosensitising compound.

The medicament may be for treating and/or preventing conditions caused by microorganisms in the oral cavity of a subject. For example, the medicament may be for treating and/or preventing periodontal and/or halitosis conditions. For example, the conditions include, but are not limited to, gingivitis, periodontitis dental caries, root caries, root canal infection, apical periodontitis and the like. The medicament may also be for managing bacteria deep within dental caries lesions. The medicament may also be used to eliminate bacterial biofilm in any localised infection.

The present invention also provides a method of treating and/or preventing conditions caused by microorganisms in a subject, wherein the method comprises the steps of:
(a) administering a photosensitising composition of any aspect of the present invention; and
(b) irradiating the area to which the composition is administered with light at a wavelength absorbed by a photosensitising compound.

The method may be for treating and/or preventing conditions caused by microorganisms in the oral cavity of a subject. For example, the method may be for treating and/or preventing periodontal and/or halitosis conditions. For example, the conditions include, but are not limited to, gingivitis, periodontitis dental caries, root caries, root canal infection, apical periodontitis and the like. The method may also be for managing bacteria deep within dental caries lesions. The method may also be used to eliminate bacterial biofilm in any localised infection.

The photosensitising compound may be comprised in the photosensitising composition. The irradiation of step (b) may be carried out for a time period of 30 minutes or less. For example, the irradiation of step (b) may be carried out for 10 seconds to 30 minutes. The time period for carrying out the irradiation of step (b) depends on the type of photosensitising compound used and the type of light used. In particular, the irradiation of step (b) may be carried out for a time period of 5 minutes to 15 minutes. Even more in particular, the irradiation of step (b) is carried out for a time period of 10 minutes.

The dose of light used in step (b) may range from 10 J/cm² to 200 J/cm². In particular, the dose of light used in step (b) ranges from 50 J/cm² to 150 J/cm².

The light used in step (b) may have any suitable wavelength. For example, the wavelength of the light depends on the type of photosensitising compound's absorbance maxima. The wavelength of the light may range from the visible to the near infra-red range of wavelength. The light used in step (b) may have a wavelength ranging from about 400 nm to about 1400 nm. In particular, the light use may have a wavelength ranging from about 600 nm to about 900 nm. In particular, the light used in step (b) has a wavelength ranging from about 650 nm to about 800 nm. Even more in particular, the light used in step (b) has a wavelength of 660 nm.

The present invention also provides a kit for treating and/or preventing conditions caused by microorganisms in a subject, the kit comprising a photosensitising composition according to any aspect of the invention, disposed in at least one suitable container. The kit may be for treating and/or preventing conditions caused by microorganisms in the oral cavity of a subject. For example, the kit may be for treating and/or preventing periodontal and/or halitosis conditions. For example, the conditions include, but are not limited to, gingivitis, periodontitis dental caries, root caries, root canal infection, apical periodontitis and the like. The kit may also be used for managing bacteria deep within dental caries lesions. The kit may also be used to eliminate bacterial biofilm in any localised infection.

The kit may further comprise at least one light emitting device capable of emitting light at a wavelength absorbed by a photosensitising compound. The photosensitising compound may be comprised in the photosensitising composition.

Another aspect of the present invention is a method of preparing the composition according to any aspect of the invention. The method may comprise the steps of: (a) preparing a mixture by mixing: a least one surfactant; at least one alcohol; and/or water, with the proviso that when the at least one surfactant is an alcohol, it is different from the at least one alcohol. The method may further comprise the step of adding at least one photosensitising compound to the mixture of step a).

### BRIEF DESCRIPTION OF THE FIGURES

FIG. 1 shows the % reduction of bacteria in the six specimens of Example 1.

FIG. 2 shows the number of cells remaining in samples containing different photosensitising compositions after PDT, with and without light.

FIG. 3 shows the effect of PDT using different photosensitising compositions.

FIGS. 3A and 3B refer to controls. FIGS. 3C and 3D show the results obtained using a photosensitising composition comprising mineral oil, an oxygen carrier and methylene blue, the composition irradiated under direct light.

FIG. 4 shows the absorption spectrum of 50 μM methylene blue in different formulations.

FIG. 5 shows the monomer to dimmer ratio of increasing concentration of methylene blue in different formulations of photosensitising composition.

FIG. 6 shows the fluorescent intensity of increasing concentrations of methylene blue in different formulations of photosensitising composition.

FIG. 7 shows a graph of % diffusion of methylene blue in different formulations of photosensitising composition across the dentinal tubules and to the apical foramen of the tooth specimens.

FIG. 8 shows a graph of % dye uptake of methylene blue in different formulations of photosensitising composition by bacterial cells, E. faecalis is a gram positive bacteria and A. actinomycetemcomitans is a gram negative bacteria.

FIG. 9 shows a graph of the log number of E. faecalis and A. actinomycetemcomitans cells in biofilm surviving the PDT.

FIG. 10 shows a graph of log number of E. faecalis and A. actinomycetemcomitans cells in biofilm in root canal dentine, surviving the PDT.

FIG. 11 shows the diffusion of methylene blue into dentine under different formulations.

FIG. 12 shows the percentage of cells surviving after PDT using Indocyanine Green.

FIG. 13 shows the percentage of cells surviving after PDT using methylene blue.

### DETAILED DESCRIPTION OF THE INVENTION

Bibliographic references mentioned in the present application are for convenience listed in the form of a list of references and added at the end of the Examples. The whole content of such bibliographic references is herein incorporated by reference.

As mentioned above, persistence of bacteria within the root canal dentine after RCT is the main cause of failure in root canal treatment. The factors that contribute to the persistence of bacteria are: (a) anatomical complexities in the root canal system; (b) porous nature of the dentine structure; (c) microbial rectors; and (d) clinical factors.

(a) Anatomical Complexities in the Root Canal Systems

The complex anatomy of the root canal system routinely makes thorough disinfection of bacteria from the root canal almost Impossible.

(b) Porous Nature of the Dentine Structure

Dentine is a tubular or porous hard tissue that forms the major bulk of human tooth. Its characteristic porous structure is attributed to tubular structures called dentinal tubules that traverse the bulk of the dentine. The number of dentinal tubules/mm² in dentine varies from 15,000 per mm³ at the outer aspect of the dentine to 45,000 mm³ at the inner aspect of the dentine (near the root canal or pulp). A dentinal tubule is normally larger in diameter than an average oral streptococcal cell, which is 0.5-0.7 μm in diameter.

(c) Microbial Factors

Different in vitro experiments have demonstrated bacterial invasion of dentinal tubules when pure cultures of Streptococcus gordonii, Streptococcus sanguis, Porphyromonas species, Actinomycetes naeslundi, E. faecalis and Capnocytophaga gingivalis, as well as bacterial plaque from periodontal lesions, are grown on human dentine (Love R M, 1998; Berkiten M et al, 2000; Perez et al, 1993a). Perez et al noted that the penetration depth of Streptococcus sanguis into the dentinal tubules of bovine incisors is 434.8 μm at the end of 20 days, whereas the studies by Berkiten M et al showed a penetration depth of 382.3 μm for Streptococcus sanguis, and 25.9 μm for Prevotella intermedia. All these studies vividly highlight active bacteria invasion and survival within dentinal tubules (Berkiten M, 2000; Perez F, 1993b; Sundqvist G, 1992; Hancock H H et al. 2001).

Bacterial floras in endodontic infections are considered mixed in nature consisting of both aerobic and anaerobic bacteria, with a predominant existence of anaerobic bacteria. Recent researches have revealed a distinct shift in the bacterial flora from a “poly microbial infection” in necrotic root canals to a “mono infection” in a falling endodontically treated teeth. This distinct difference in the endodontic bacterial flora is attributed to the Endodontic Environmental Transition (EET). Hancock et el has associated the EET to the difference in selective pressures and oxygen tension that exists in the necrotic tooth material versus those in the previously treated root canals (Marsh P D, 1994).

Biofilm mode of growth is a survival strategy and harsh environmental conditions existing in the root canal may favour the growth of bacteria as a biofilm in root canal. This aspect is supported by the fact that, clinically isolated bacteria possess increased adhering capacity, increased virulence factors such as capsular polysaccharides, gelatinase, and increased resistance to antimicrobials, which are all characteristics of biofilm style of growth (Stewart P S, 1996; Mah T F C and O'Toole G A, 2001; Michael R W et al, 2001). When bacteria grow as biofilm, the altered genetic and metabolic processes along with its complex matrix prevent the action of antimicrobial agents. Subsequently, the colonizing organism gains protection against unfavourable environmental conditions (Stewart P S and Costerton J W, 2001). The antibiotic resistance has been found to increase up to 1000-1500 times when bacteria are grown in biofilm, compared to planktonic cells. Further, there is also a constant detachment of ceils from a fully matured biofilm, and the detached cells serve as a steady source for chronic infection. Thus, bacterial biofilm is associated with a wide range of persistent infections in human beings. Interestingly, clinical examination of root tips of teeth associated with refractory periapical periodontitis, have suggested the presence of bacterial biofilm in the apical portion of the root canal and the extraradicular region (Tronstad L and Sunde P T, 2003; Noiri Y et al, 2002).

(d) Clinical Factors

In the root canal treatment of infected teeth, clinicians utilise an instrumented cleaning procedure, supplemented with intra-canal irrigants and medicaments to achieve root canal disinfection. Irrigants most commonly used for this are sodium hypochlorite, chlorhexidine and Tubulicid (0.2% EDTA), while calcium hydroxide and camphorated paramonochlorophenol are used as root canal medicaments. The effectiveness of the endodontic irrigants at various dentinal tubular depths is not very clear as yet. It is demonstrated that bacteria remains viable at greater depths from the pulp at all levels in the root canal. In addition, long terms use of such root canal medicaments may lead to the development of resistance in the target organisms. Is has been shown that treatment of dentine with caustic chemicals such as sodium hypochlorite may alter the physical and chemical characteristic of dentine substrate. However, a longer duration of contact and mechanical instrumentation is required for these chemicals to be effective against endodontic microbes. Adding to this, certain species of bacteria show conspicuous tendency to penetrate or invade the dentinal tubules, which is much higher than the effective-penetration-depth of various chemicals used in endodontic therapy.

Accordingly, there is a need of an alternative approach to eliminate microbial flora from root canal and dentinal tubules of teeth. This alternate technique should be able to: (1) be non-invasive (produce non-trauma to patient without undue removal of dentine structure), easy to practice and produces immediate killing of microbes; (2) eliminate the total bacterial flora (aerobic, anaerobic, Gram-positive, Gram-negative) and/or biofilms in root canal systems and within dentinal tubules; and (3) reduce bacterial population in the periapical region (adjacent to the apical foramen).

Recently, the use of photodynamic therapy (PDT) to eliminate bacteria has gained much interest among researchers from the academic and clinical field, in principle, PDT involves the killing of microorganisms, when a photosensitiser selectively accumulates or present as endogenously produced, is activated by a specific wavelength of light to produce deleterious chemical entities of oxygen free radicals.

A type I process generates reactive free radicals, peroxides and superoxides by electron or hydrogen transfer reaction with water or with a biomolecule to produce a cytotoxic result. In a type II process, the excited triplet state of the photosensitiser reacts with the oxygen in the tissue and converts the oxygen molecule from the normal triplet state form to a highly reactive excited singlet state form, it is the type II process that is generally accepted as the major pathway for photodynamic therapy.

The pathway in which a photosensitiser triplet state reacts first with a substrate other than molecular oxygen is termed Type I. In a Type I reaction, a donor-acceptor electron transfer takes place from the photosensitizer to the substrate forming active free radicals. In the alternate Type II pathway, the photosensitizer triplet state reacts with molecular oxygen. Type II photosensitization of a biological system is referred to as photodynamic action. In a Type II reaction, the photosensitiser (dye) absorbs light energy of specific wavelengths, produces a radiation which causes energy transition from the dye triplet state to molecular oxygen activating strong reactive oxygen. The resultant active oxygen, which is an unstable, and energy rich active oxygen, is called the singlet molecular oxygen. The singlet molecular oxygen is responsible for the oxidation process in a photodynamic reaction.

The advantage of using PDT is that it is capable of total elimination of microbes (i.e. bacteria (aerobic, anaerobic, Gram positive, Gram negative) and viruses). Further, PDT does not necessitate excessive removal of tooth structure and therefore, prevents post-treatment weakening of tooth. Application of caustic chemicals such as sodium hypochlorite or EDTA on dentine will affect the mechanical properties of the dentine. However, PDT minimises such deleterious effects on the mechanical properties of the dentine. Further, the time span for routine endodontic procedure can be reduced. Other advantages are as follows: development of drug resistant bacterial strains can be evaded; antibiotic sensitivity spectrum and chemical resistance of bacteria is no longer a major issue; no cumbersome or expensive equipments are involved; and the method is easy to use, which can be developed to an efficient and cost effective treatment option.

At present, there are certain limitations of PDT in disinfecting the root canal dentine. Some of the challenges faced are as described below:

(a) Root Canal Microenvironment

The reduced oxygen tension in the root canal, especially at the root apex, will reduce the effectiveness of the PDT since the antimicrobial activity is mediated through reactive oxygen species.

(b) Optical Scattering in Dentine Tissue

Dentine tissue scatters light (both forward and backward scatter) and also absorbs light. The light-dentine tissue interaction will effect light conduction through dentine and consequently cause significant decline in the light energy (and/or dosimetry).

(c) Penetrability of Photosensitiser Into the Complexities of the Root Canal and Dentinal Tubules

It is important to consider an appropriate vehicle to facilitate better diffusion of the photosensitiser in the root canal complications (ramifications such as fins, isthmuses blacked canals) and to the apical foramen (terminus of the root canal).

(d) Binding of Photosensitiser with Bacterial Cells

From the point of view of localisation in cells, the best photosensitisers are those that are hydrophobic in order for them to penetrate cell membranes most readily. However, to allow the photosensitiser to penetrate the dentinal tubules and anatomical complexities, they have to be hydrophilic.

Therefore, an ideal photosensitising composition or formulation for PDT in root canal infection should allow: (a) penetration of the photosensitiser deep into the dentinal tubules and anatomical complexities of the root canal; (b) improve the photophysical characteristics of the photosensitiser; (c) supply adequate amount of oxygen; (d) minimise scattering of light by dentine; and (e) enables conduction or transmission of light with minimal energy loss deep into the dentine tissue.

In formulating an improved photosensitising composition, several conditions will have to be taken into consideration: (1) photodynamic therapy (PDT) and/or light activated therapy; (2) the exploitation of the structural and optical characteristics of the dentine; and (3) creation of a liquid wave-guide within the lumens of the root canals and dentinal tubules. This would produce maximum light conduction through the artificial liquid wave guide to greater depths in the dentine. Also, biologically inert chemicals that enhance the quantum yield of active oxygen species to achieve deeper kill of sensitized bacterial cells may be utilised. Hydrophilic and/or surfactant medium (with both hydrophilic and hydrophobic functional groups) to transport hydrophobic and/or cationic photosensitisers to greater depth into anatomical complexities and dentinal tubules, as well as, agents such as polymers that enhance the cellular uptake of photosensitiser (L- or D-arginine) and/or render photosensitiser binding specific to prokaryotic cells may be utilised. Further, if endogenous pigments of bacteria are present, these may also be utilised as photosensitisers for the purposes of the present invention.

The present invention solves the problems of the prior art, and provides a new photosensitising composition and its uses thereof. In particular, there is provided a photosensitising composition comprising a mixture of: at least one surfactant, at least one alcohol, and optionally water. The composition may further comprise at least one photosensitising compound.

According to one aspect, the present invention provides a photosensitising composition comprising a mixture of: at least one surfactant; at least one alcohol; and/or water, with the proviso that when the at least one surfactant is an alcohol, it is different from the at least one alcohol. In particular, the present invention provides a photosensitising composition comprising a mixture of: at least one surfactant: at least one alcohol; and/or water, wherein the ratio of the volume of the at least one surfactant to the at least one alcohol to water is in the range of 10:5:85 to 40:30:30, with the proviso that when the at least one surfactant is an alcohol, it is different from the at least one alcohol. The volume ratio may be in the range of 15:10:75 to 35:25:40. Even more in particular, the ratio is 30:20:50.

Any suitable surfactant may be used for the present invention. For example, the at least one surfactant may be a trihydric alcohol or a polyester. The at least one surfactant may be a refractive index matching liquid. The polyester may be polyethylene glycol or polypropylene glycol. The trihydric alcohol may be glycerol. Examples of surfactants that may be used for the present invention include, but are not limited to, mineral oil, glycerol, polyethylene glycol, Triton X, polypropylene glycol, SDS and any detergent suitable for use for the present invention. If the surfactant is an alcohol, the alcohol is different from the at least one alcohol.

The use of surfactants may improve the penetrability of photosensitisers into the complexities of the root canal and dentinal tubules. For example, the use of hydrophilic and/or surfactant containing medium may improve the use of the photosensitising composition in PDT. Surfactants facilitate penetrability of the photosensitizing composition into the root canal complications and reach the apical foramen of the tooth. The surfactants reduce the surface tension and therefore, enhance the penetrability of the photosensitising solution. Further, hydrophobic photosensitisers can be more easily dissolved in a combination of polyethylene glycol (PEG), ethanol and water, as provided by the present invention, to improve its penetrability, and therefore its use as a photosensitiser in PDT.

The photosensitising composition of the present invention may also comprise a refractive index matching liquid. In particular, the composition comprises a high refractive index liquid medium. The refractive index liquid medium may create a liquid wave guide in the dentine. The unique structural and optical characteristics of the dentine tissue is utilised in combination with a high refractive index liquid medium such as glycerol or mineral oil to achieve a liquid optical-conduit (waveguide)-effect in root canal lumen and dentinal tubules. The optical-waveguide-effect will aid in diminishing dentine tissue scatter and in addition, the higher refractive index of the medium, when compared to the dentine, will enable achieving predominantly total internal reflection and better light energy distribution within the root canal lumen (and anatomical complexities) and the dentinal tubules. High refractive index liquids such as glycerol and mineral oil minimise tissue scatter and obtain total internal reflection within the root canal lumen and dentinal tubules. Therefore, a suitable liquid, or mixtures thereof, will be one that can provide ideal refractive index to transform root canal lumen and dentinal tubular spaces into optical conduit.

Any suitable alcohol may be used for the present invention. For example, the alcohol may be a monohydric alcohol. The alcohol may also be a dihydric alcohol and/or a trihydric alcohol. A monohydric alcohol contains only one hydroxyl group in each molecule. Examples of monohydric alcohols include, but are not limited to, methanol, ethanol, propanol, butanol, pentanol, hexacecanoyl, melissyl alcohol and the like. Further, the monohydric alcohol may be a primary, secondary or tertiary alcohol. Examples of dihydric alcohols include glycols and dials. Examples of trihydric alcohols include glycerol. In particular, the alcohol is ethanol.

According to a particular aspect of the present invention, composition comprises a mixture of polyethylene glycol, ethanol and water. According to another particular aspect, the composition comprises a mixture of glycerol, ethanol and water. The ratio of the volume of glycerol to ethanol to water in the mixture may be in the range of 10:5:85 to 40:30:30. In particular, the ratio is in the range of 15:10:75 to 35:25:40. Even more in particular, the ratio is 30:20:50.

The composition of the present invention may further comprise at least one photosensitising compound. The at least one photosensitising compound comprised in the photosensitising composition may be any suitable photosensitising compound. For the purposes of the present invention, the terms ‘photosensitising compound’ and ‘photosensitiser’ may be used interchangeably. A suitable choice of a photosensitiser should preferably have certain characteristics. The photosensitiser must have the ability to selectively accumulate in target areas, for example, cancerous and/or pre-cancerous tissues. In other words, while it is eliminated from normal tissue, it is retained in cancerous tissue and/or pre-cancerous cells. Further, from the point of view of localisation in target areas, for example in periodontal areas or in tumours, the best photosensitisers are those that are hydrophobic in order for them to penetrate cell membranes more readily. However, if the photosensitisers are to be administered intravenously, the photosensitisers should be at least partially water soluble and therefore, also hydrophilic, to disperse in the blood stream. Therefore, combining the two requirements, it is preferable to use a photosensitiser which is amphiphilic. Alternatively, the photosensitiser may be modified to have amphiphilic properties by chemically modifying a fundamentally hydrophobic photosensitiser by attaching polar residues such as amino acids, sugars and/or nucleotides.

The photosensitiser should also be capable of absorbing light at a wavelength in the in the region of maximum transparency of biological tissues. This would allow light to penetrate deeper in the tissue to activate the photosensitiser. This is particularly useful if the target area is deep and it is desired for the photosensitiser to roach the target area effectively. For example, malignant tissues which are deep would require the photosensitiser to absorb light at a long wavelength. However, wavelengths longer than 900 nm are energetically too low to provide sufficient energy required for the excitation of triplet oxygen to its singlet state in PDT.

A suitable photosensitiser should also be able to exhibit minimum toxicity in the dark in order for light activation of the drug to produce maximum benefits without side effects derived from the inherent toxicity. Further, the photosensitiser should have a high yield of triplet-state formation and a long triplet lifespan. In other words, the non-radiative intersystem crossing from the excited singlet state of the photosensitiser to its excited triplet state should be efficient compared to the direct radiative transition (fluorescence) from the excited singlet state. A longer triplet lifespan would enhance the chance of producing a cytotoxic reagent or a cytotoxic reaction from the excited state.

The photosensitiser should also not aggregate since aggregation can reduce the extinction coefficient and shorten the lifespan and quantum yield of the excited triplet state. Aggregated forms of photosensitiser can also affect its pharmacokinetics and biodistribution. The photosensitisor should also be able to rapidly excrete from the body of the subject it is administered to. This will produce low systemic toxicity and will reduce sunlight sensitivity following PDT.

Examples of PDT include porphyrin derivatives. The first group of photosensitisers used in clinical PDT was hematoporphyrin derivatives. Photofrin® (herein referred to as Photofrin), a photosensitiser obtained from hematoporphyrin by treatment with acids is approved by the U.S. Food and Drug Administration, as well as by other regulatory agencies throughout the world for the treatment of a variety of malignant tumours. Photofrin is actually a complex mixture consisting of various derivatives, as well as dimeric and oligomeric fractions. In commercial Photofrin, the fractions are partly purified to be around 85% oligomeric materials. Because Photofrin is a complex mixture, there are still concerns about the identity of the active components and the reproducibility of the synthetic process producing it Photofrin is a non-toxic drug. However, the disadvantage is that it is retained for some time by the skin. For this reason, patients are required to avoid direct sunlight, very bright artificial lights or strong residential indoor lighting for a period of 4 to 6 weeks after injection of the drug.

In order to prepare “second-generation” photosensitisers that consist of pure single components (as opposed to a mixture comprising Photofrin), and be capable of absorbing light at a wavelength further in the red region to provide deeper penetration in tissues, efforts have already led to many promising compounds for use as photosensitisers. These include modified porphyrins, chlorins, bacteriochlorins, phthatocyanines, naphthocyanines, pheophorbides, and purpurins (Dougherty et al, 1998; Dotty M R et al, 2004).

Chlorins and bacteriochlorins are attractive because of their ability to absorb longer wavelength. However, these classes of drugs undergo re-aromatisation of the pyrrole rings to produce porphyrins, which limit their lifetime in vivo as photosensitisers. Further, none of these classes of materials have been FDA approved specifically for cancer treatment.

Another example of a photosensitiser is 5-Aminolaevulinic Acid (ALA). ALA is a metabolic precursor in the biosynthesis of hematoporphyrin, which endogenously generates an effective photosensitiser, protoporphyrin IX. It thus provides an alternative to the administration of an exogenous photosensitiser. Even though ALA can be endogenously generated from glycine and succinyl CoA, exogenous administration of ALA is chosen for a controlled build-up of protoporphyrin IX. The advantages offered by ALA-induced protoporphyrin IX over Porphyrin are: (1) ability to reach optimum therapeutic ration in 4-6 hours; (2) rapid systemic clearance of the photosensitiser within 24 hours, thus not only eliminating prolonged skin photosensitivity, but also allowing repeated treatment every 24 hours; and (3) accurate analysis of photosensitiser levels by in situ monitoring of its fluorescence. However, the limitation of ALA stems from its hydrophilic nature, which restricts its penetration through keratinous lesion of normal skin. For this reason lipophilic ALA esters may be preferable because they can penetrate cells more readily.

Yet another type of photosensitisers are phthalocyanines and naphthalocyanines. These are another class of PDT photosensitisers that absorb light in the long wavelength region of between 670 nm and 780 nm, and exhibit high molar extinction coefficient. These photosensitisers are hydrophobic in nature and exhibit limited solubility. Their solubility can be enhanced by attaching sulfonic acid, carboxylic acid or amino acid groups to the ring. Clinical evaluation of sulfonated phthalocyanine for use in PDT has been further motivated by its negligible dark toxicity, its minimal skin photosensitivity and its ability to be photoactivated at a much longer wavelength. The phthalocyanines and naphthalocyanines are already in the early stages of preclinical and clinical evaluations. However, a problem encountered with these compounds is their tendency to aggregate in aqueous media at relatively low concentrations, resulting in a loss of their photoactivity.

Cationic photosensitisers are also suitable. This class of photosensitisers carries a positive charge on the heteroatom of the ring structure. These cationic PDT photosensitisers tend to be bound intracellularly. Another distinction is that some of these photosensitisers (e.g. rhodamine 123 (Rh-123)) are selectively taken up by the mitochondria of living cells. Methylene blue (basic dye) is a cationic photosensitiser, which is currently in clinical use. It is important to note that the surfaces of bacterial cells are negatively charged and basic photosensitisers or dyes, which are positively charged, are most often used in staining cells in bacteriology.

For the purposes of the present invention, any suitable photosensitiser may be used. The photosensitising composition of the present invention may comprise at least one photosensitising compound. For example, the at least one photosensitising compound may be selected from the group consisting of: toluidene blue, methylene blue, arianor steel blue, tryptan blue, crystal violet, azure blue cert, azure S chloride, azure 2, azure A chloride, azure B tetrafluoroborate, thionin, azure A eosinate, azure B eosinate, azure mix sicc, azure it eosinate, haematoporphyrin HCl, haematoporphyrin ester, aluminium disulphonated phthalocyanine, chlorins, photoactive fullerenes (e.g. C16-b), aminolevulinic acid (ALA), bacteriochlorins, phthalocyanines, pheophorbides, purpurins, naphthalocyanines, indocyanine green, or mixtures thereof. In particular, the at least one photosensitising compound is methylene blue.

According to a particular aspect, the present invention provides a photosensitising composition comprising at least one photosensitising compound and a mixture of glycerol, ethanol and water. According to another particular aspect, the present invention provides a photosensitising composition comprising at least one photosensitising compound and a mixture of polyethylene glycol, ethanol and water.

The photosensitising composition of the present invention may further comprise other compounds in order to make the composition more suitable for use as a photosensitising composition in PDT. The photosensitising composition of the present invention may be adapted to better eliminate bacteria and/or microorganisms, in particular, the photosensitising composition may be better suited for eliminating bacteria and/or microorganisms within the anatomical complexities of the root canals and reach deeper into the dentinal tubules. Accordingly, the photosensitising composition may further comprise at least one of the following:
(a) an oxygen carrier;
(b) a polycationic compound; or
(c) an oxidising agent,
   or mixtures thereof.

Since the root apex and greater depth of dentinal tubules is considered as being a hypoxygenic physiologic site, oxygen carriers such as perfluorocarbons (PFCs) are added to the photosensitising composition to increase the performance of PDT. PFCs are chemically and biochemically inert due to the strong intra-molecular bonding (C—F bonds are 485 kJ/mol, that is 84 kJ/mol more than 3 regular C—H bond). The chemical structure and the weak intermolecular interactions are responsible for the specific properties of PFCs, namely the low surface tensions (<20 mN/m), dielectric constants and refractive indices, the high densities, viscosities and gas solubility that are the largest known for liquids. At present, PFCs are used in tissue oxygenation fluids (blood substitutes, oxygen therapeutics), as anti-tumour agents, perfusates for isolated organs, surgical tools for ophthalmology, lubrication and cushioning for particular disorders, as cell culture media supplements and in drug formulations and delivery. Any other suitable type of oxygen carriers may be used for the present invention. For example, hydroperfluoro carbons, perfluoro carbons or a mixture thereof. Particular examples include, but are not limited to, perfluorodecahydro naphthalene, perfluorodecalin, perfluorohexane, octafluoropropane, perfluorabutane, perfluorooctane and perfluoromethyldecalin. In particular, perfluorodecahydro naphthalene is added to the photosensitising composition of the present invention.

Limitation in photosensitiser uptake by microbial cells is a potential problem associated with PDT mediated bacterial killing. The limited killing of gram negative bacteria has been associated with the presence of outer membrane that acts as a functional and physical barrier between the cell and the surrounding environment. However, use of polycationic compounds such as poly L-Lysine, either coupled with the photosensitiser or co-administered, can facilitate the movement of the photosensitiser across the outer membrane of the gram negative bacteria. Polycationic compounds interact with divalent cation-binding sites on cell surface lipopolysaccharides (LPS) and displace these ions. This disrupts the normal barrier property of the outer membrane causing transient ‘cracks’ which permits passage of hydrophobic compounds such as photosensitisers. Accordingly, in order to improve the binding of photosensitisers with bacterial cells, it may be required to attach charged, hydrophobic and/or polymers to the photosensitisers.

The addition of polycationic compounds to the photosensitising composition of the present invention may further improve the performance on the photosensitising composition in PDT. Hydrophobic and cationic photosensitisers are found to bind well with bacterial cells. Interestingly, when PDT is conducted against bacteria in the presence of mammalian cells, the mammalian cells are not affected by the PDT, killing only bacteria at lower concentrations of photosensitiser (Soncin M et al, 2002). Further, the selectivity of bacteria can be improved by coupling the photosensitiser to a polypeptide chain of lysine, which can target bacterial cells (Soukos N S et al, 1998) (Gram negative and Gram positive) bearing negative charges on the outer surface. Since the mammalian cells take up macromolecule such as polypeptide by endocytosis, a temporal selectivity may be achieved if photosensitisation is performed for a shorter duration. Examples of polycationic compounds that may be used for the present invention include, but are not limited to, cationic polypeptides such as poly L-lysine, L-arginine, D-arginine, and multivalent cations such as calcium chloride, calcium hydroxide and magnesium chloride (Soukos N S et al, 1998).

The composition of the present invention may also comprise an oxidising agent. The advantage of using an oxidising agent is that it may pre-activate the photosensitising compound, and hence shorten the duration of the PDT (Pervaiz S, 2001). Any suitable oxidising agent may be used for the present invention. Examples of oxidising agents which may be used include, but are not limited to, dilute sodium hypochlorite, hydrogen peroxide, DMSO and chlorine dioxide.

It has been found that photoproducts formed by irradiating the photosensitiser (outside biological sites) have a long lifespan and when such pre-activated photosensitiser is applied to the actual site (in the subject's body), it produces a better outcome. This type of photodynamic therapy is termed ‘pre-activation’ or ‘pre-irradiation therapy’ (Pervaiz S, 2001). The action is mediated by the photoproducts formed from the photosensitiser on irradiation which depends on the physical-chemical conditions under which the irradiation is done. This includes nature of the photoproducts, wavelength(s) used, intensity of light, temperature, oxygen and duration of irradiation. The specificity of photo products to the targets as the parental compound promises a better treatment outcome. This is of particular use when applied to tooth (dentine) since scattering and absorption of light may diminish the dosimetry of light.

According to another aspect of the present invention, it provides for the utilisation of endogenous pigments of bacteria as the at least one photosensitising compound. For example, many obligatory anaerobes are found to have endogenous pigment (e.g. Porphyromonas species, Bacteroides species) such as porphyrin. These bacterial pigments may be utilised as endogenous photosensitisers to achieve the killing of the bacteria during PDT. In this approach only optimum light energy at specific wavelength is required. An additional photosensitiser (exogenous photosensitiser) is not required. These groups of bacteria are common and dominant group in root canal infections, particularly in the apical region of the root canal (deeper aspect close to the root tip). Accordingly, a photosensitising compound may not be required to be included in the photosensitising composition.

The composition according to the present invention may further comprise a pharmaceutically or pharmacologically acceptable excipient, diluent and/or carrier. The phrase “pharmaceutically or pharmacologically acceptable” refers to molecular entities and compositions that do not produce an adverse, allergic or other untoward reactions when administered to an animal, or a human, as appropriate. The compositions of the present invention may be an aqueous composition, optionally comprising an effective amount of the photosensitising compound, dissolved or dispersed in a pharmaceutically acceptable carrier or aqueous medium. An example of a suitable carrier includes water, such as distilled water or demineralised water, preferably pyrogen-free, sterile water or water for injection. The composition may additionally comprise buffers, salts for adjusting the tonicity of the composition, preservatives, gelling agents and the like. The use of such agents for pharmaceutical active substances is well known in the art.

The composition of the present invention may be formulated according to its means of use and/or administration. For example, the composition may be formulated for use in the treatment and/or prevention of conditions caused by microorganisms in a subject. The composition may be formulated for use in the treatment and/or prevention of conditions caused by microorganisms in the oral cavity of a subject. The composition may be formulated for the treatment and/or prevention of conditions such as periodontal and/or halitosis conditions.

The composition may also be formulated for use in oral cavity treatment in a subject. The subject may be an animal or human. The composition may be formulated such that it is accessible to the interior surfaces of the mouth, including the tongue, buccal mucosa and/or gum regions. The composition may be formulated such that it is suitable for administering the composition topically or by injection. The composition may also be formulated as an oral rinse, a mouthwash and/or an atomizing spray.

According to another aspect, the composition according to the present invention may be for use in medicine. For example, the composition may be used for the treatment and/or prevention of conditions caused by microorganisms in a subject. The composition may be used for oral cavity treatment in a subject. In particular, the composition may be used for the treatment of microorganisms in the oral cavity of a subject. The composition may also be used for the prevention of microorganisms in the oral cavity of a subject. The subject may be an animal or human. The composition may also be used for the treatment and/or prevention of conditions caused by microorganisms in the oral cavity of a subject. Examples of such conditions include, but are not limited to, periodontal conditions and halitosis conditions. For example, the conditions include, but are not limited to, gingivitis, periodontitis dental caries, root caries, root canal infection, apical periodontitis and the like. The composition may also be used for managing bacteria deep within dental caries lesions. The composition may also be used to eliminate bacterial biofilm in any localised infection.

As stated above, the present invention is useful for the elimination of a broad spectrum of microorganisms found within the dental tissues. This mode of microbial elimination is very important keeping in mind the pores nature of dentine (because of the dentinal tubules) and the ability of microbes to penetrate into these dentinal tubules. Other major limitations associated with conventional treatment, which are circumvented in the present invention are: (1) limited penetration of chemicals into these porosities or dentinal tubules; (2) ability of precipitated calcium from the dental hard tissue to buffer the efficacy of the chemical disinfectant; (3) the anaerobic environment prevailing within the hard tissues (such dentinal tubules), which can diminish the efficacy of the photodynamic therapy; and (4) the ability of bacteria to survive in a “highly drug resistant” biofilm state inside these locations.

The photosensitising composition according to any aspect of the present invention is capable of achieving deeper diffusion of the primary photosensitising compound, such as methylene blue, into the dental tissue, such as dentine. The composition is designed to have maximum uptake of the primary photosensitising compound by the bacterial cells and minimal aggregation of the primary photosensitising compound within the medium. The photosensitising composition may further comprise a liquid conduit (LC). The liquid conduit may be added to the photosensitising composition as a separate step after a period of irradiation to minimise light scattering (found within the dentine tissue) achieve greater penetration of light energy and effective killing of bacteria. The conduit is chosen in such a way that they are transparent liquid, low refractive index (similar or slightly less than water), inert, immiscible with water (to prevent atomic absorption and less light energy loss) and can be a good source for free oxygen radical to facilitate killing of bacteria and bacterial biofilm deep with in the dentine tissue. For example, perfluoro carbon compounds may be used as liquid conduits. Perfluoro carbon compounds have desirable optical quality and low absorption of light (UV-VIS-IR). They have good thermodynamic property (reduced surface tension, viscosity) and desirable chemical stability. Other important features of this compound include: (1) lack of biological activity; (2) short retention time in the body; (3) their ability to dissolve gas (especially oxygen and carbon dioxide); (4) they have antimicrobial (Economou-Stamatelopoulou C et al, 2003) and anti-inflammatory effect and has been used for wound healing (J D Whitney, 1989); and (5) facilitate better light energy delivery (Yoshida H et al, 1997).

Another aspect of the present invention is a use of the photosensitising composition as described above in the manufacture of a medicament for treating and/or preventing conditions caused by microorganisms in a subject. The subject may be an animal or a human. The treatment and/or prevention may comprise the steps of:
(a) administering the photosensitising composition according to any aspect of the present invention; and
(b) irradiating the area to which the composition is administered with light at a wavelength absorbed by a photosensitising compound.

The medicament may be for treating and/or preventing conditions caused by microorganisms in the oral cavity of a subject. For example, the medicament may be for treating and/or preventing conditions caused by microorganisms, such as periodontal conditions and halitosis conditions. The conditions may include any one of the following: gingivitis, periodontitis dental caries, root caries, root canal infection, apical periodontitis and the like. The medicament may also be for elimination of bacterial biofilm in any localised infection and/or managing bacteria deep within dental caries lesions.

The photosensitising compound may be comprised in the photosensitising composition. Any suitable photosensitising compound may be used as described above.

The treatment and/or prevention may further comprise a step of waiting a predetermined period of time between steps (a) and (b) above. The step of waiting for a predetermined period of time between steps (a) and (b) may be from about 1 minute to 5 days. The waiting step may be from about 10 minutes to 3 days. The waiting step may be from 20 minutes to 1 day. The waiting step may be from 30 minutes to 5 hours. The steps described above may be repeated as often as required, for example until the condition has reduced to a desired level or has been eliminated. The steps may be repeated after intervals of predetermined periods. The interval for the repeatability of the steps would be obvious to a person skilled in the art. For example, the steps may be repeated every few hours, every day, every 2 or 3 days.

The photosensitising composition in step (a) may be administered topically. The photosensitizing composition may also be administered by injection. The composition may be administered to the interior surfaces of the mouth. Including the tongue, buccal mucosa and/or gum regions. The composition may also be administered to any part of the oral cavity which is need of such composition.

Any suitable light may be used for the irradiation in step (b). For example, a low powered light source or a diode laser source may be used. Any suitable light such as visible or infrared lasers may be used. High energy nan-visible light such as tungsten halogen or xenon arc source may also be used. LED light sources may also be used. The advantage of using LED light sources is that it will reduce the potential for the generation of uncomfortable heat, and therefore cause less discomfort to the subject. The irradiation of step (b) may be performed for the whole of the affected area, in particular, irradiation is performed for the whole interior of the mouth. For example, the light source may be manipulated such that accessible interior surfaces are irradiated. Alternatively, only some areas are irradiated. For example, individual pockets of areas may be irradiated. The light source may be adapted to irradiate all regions of the oral cavity, including under the tongue and through the flesh covered lingua), labial, anterior and posterior areas of the oral cavity and through the bite surface.

The light used in step (b) may have any suitable wavelength. The wavelength depends on the photosensitising compound used for the purposes of the present invention. The wavelength depends on the type of photosensitising compound s absorbance maxima. The wavelength of the light may range from the visible tight range to the near infrared range of wavelength. The light source may have a wavelength ranging from about 400 nm to about 1400 nm. The light source may have a wavelength ranging from about 600 nm to 900 nm. in particular, the wavelength is ranging from about 650 nm to about 800 nm. Even more in particular, the wavelength is about 660 nm, or about 664 nm. The irradiation enables the activation of the photosensitising compound. Accordingly, the wavelength used for the irradiation will depend on the photosensitising compound used for the PDT. For example, if indocyanine green (ICG) is used as the photosensitising compound, a laser light of 808 nm wavelength may be used. Further, near infrared light is expected to penetrate hard tissues batter. It should be noted that any non-toxic photosensitiser, and subsequently tight source with optimum wavelength, will be utilised to achieve light activation.

When endogenous pigments of obligatory anaerobes are used as ‘endogenous photosensitisers’ without the use of additional exogenous photosensitisers, light sources at a lower range in the visible spectrum is required. For example, if the endogenous pigment porphyrin in Porphyromonas is used as the photosensitiser in PDT, a tight of wavelength of about 400 nm would be required.

The dose of light used in step (b) may range from 10 J/cm² to 200 J/cm². In particular, the dose of light may range from 50 J/cm² to 150 d/cm².

The intensity of the light source may range from 1 to 100 mW. In particular, the intensity may range from 20 to 50 mW. Even more in particular, the intensity of the light source is about 30 mW.

The irradiation of step (b) may be carried out for any suitable period of time. For example, the irradiation is carried out for a time period of about 30 minutes or less. For example, the irradiation of step (b) may be carried out for about 10 seconds to 30 minutes. The time period for carrying out the irradiation of step (b) may depend on the type of photosensitising compound used and the type of light used. In particular, the irradiation may be carried out for a time period of about 5 minutes to 15 minutes. Even more in particular, the irradiation is carried out for about 10 minutes. The irradiation may be performed for the entire mouth of the subject or specific regions of infection, for example, at the area of caries lesion or at the root canal.

The present invention also provides a method of treating and/or preventing conditions caused by microorganisms in a subject, wherein the method composes the steps of:
(a) administering the photosensitising composition according to any aspect of the present invention; and
(b) irradiating the area to which the composition is administered with light at a wavelength absorbed by a photosensitising compound.

The method may be for treating and/or preventing conditions caused by microorganisms in the oral cavity of a subject. For example, the method may be for treating and/or preventing conditions caused by microorganisms, such as periodontal conditions and halitosis conditions. The conditions include, but are not limited to, gingivitis, periodontitis dental caries, root caries, root canal infection, apical periodontitis and the like. The method may also be for elimination of bacterial biofilm in any localised infection and/or managing bacteria deep within dental caries lesions. The subject may be an animal or a human.

The photosensitising compound may be comprised in the photosensitising composition.

The treatment and/or prevention may further comprise a step of waiting a predetermined period of time between steps (a) and (b) above. The step of waiting for a predetermined period of time between steps (a) and (b) may be from about 1 minute to 5 days. The waiting step may be from about 10 minutes to 3 days. The waiting step may be from 20 minutes to 1 day. The waiting step may be from 30 minutes to 5 hours.

The steps described above may be repeated as often as required, for example until the condition has reduced to a desired level or has been eliminated. The steps may be repeated after intervals of predetermined periods. The interval for the repeatability of the steps would be obvious to a person skilled in the art. For example, the steps may be repeated every few hours, every day, every 2 or 3 days.

The photosensitising composition in step (a) may be administered topically. For example, the photosensitising composition may administered by injection. The composition may be administered to the interior surfaces of the mouth, including the tongue, buccal mucosa and/or gum regions. The composition may also be administered to any part of the oral cavity which is need of such composition.

Any suitable light source may be used for the irradiation in step (b). For example, a low powered light source or a diode laser source may be used. Any suitable light such as visible or infrared lasers may be used. High energy non-visible light such as tungsten halogen or xenon arc source may also be used. LED light sources may also be used. The advantage of using LED light sources is that it will reduce the potential for the generation of uncomfortable heat, and therefore cause less discomfort to the subject. The irradiation of step (b) may be performed for the whole of the affected area. In particular, irradiation is performed for the whole interior of the mouth. For example, the light source may be manipulated such that accessible interior surfaces are irradiated. Alternatively, only some areas are irradiated. For example, individual pockets of areas may be irradiated. The light source may be adapted to irradiate all regions of the oral cavity, including under the tongue and through the flesh covered lingual, labial, anterior and posterior areas of the oral cavity and through the bite surface.

The light used in step (b) may have any suitable wavelength. The wavelength depends on the photosensitising compound used for the purposes of the present invention. For example, the wavelength of the light depends on the type of photosensitising compound's absorbance maxima. The wavelength of the light may range from the visible light range to the near infrared range of wavelength. The light source may have a wavelength ranging from about 400 nm to about 1400 nm. The light source may have a wavelength ranging from about 600 nm to about 900 nm. In particular, the wavelength is ranging from about 6S0 nm to about 800 nm. Even more in particular, the wavelength is about 680 nm, or about 664 nm. The irradiation enables the activation of the photosensitising compound. Accordingly, the wavelength used for the irradiation will depend on the photosensitising compound used for the PDT. For example, if indocyanine green (ICG) is used as the photosensitising compound, a laser light of 808 nm wavelength may be used. Further, near infrared light is expected to penetrate hard tissues better, it should be noted that any non-toxic photosensitiser, and subsequently light source with optimum wavelength, will be utilised to achieve light activation.

When endogenous pigments of obligatory anaerobes are used as ‘endogenous photosensitisers’ without the use of additional exogenous photosensitisers, light sources at a lower range in the visible spectrum is required. For example, if the endogenous pigment porphyrin in Porphyromonas is used as the photosensitiser in PDT, a light of wavelength of about 400 nm would be required.

The dose of light used in step (b) may range from 10 J/cm² to 200 J/cm². In particular, the dose of light may range from 60 J/cm² to 150 J/cm².

The intensity of the light source may range from 1 to 100 mW. In particular, the intensity may range from 20 to 50 mW. Even more in particular, the intensity of the light source is about 30 mW.

The irradiation of step (b) may be carried out for any suitable period of time. For example, the irradiation is carried out for a time period of about 30 minutes or less. For example, the irradiation of step (b) may be carried out for about 10 seconds to 30 minutes. The time period for carrying out the irradiation of step (b) may depend on the type of photosensitising compound used and the type of light used. In particular, the irradiation may be carried out for a time period of about 5 minutes to 15 minutes. Even more in particular, the irradiation is carried out for about 10 minutes. The irradiation may be performed for the entire mouth of the subject or specific regions of infection, for example, at the area of caries lesion or at the root canal.

The present invention also provides the cosmetic non-therapeutic method of the treatment, elimination or prevention of microorganisms in a subject, the method comprising the steps of:
(a) administering the photosensitising composition according to any aspect of the present invention; and
(b) irradiating the area to which the composition is administered with light at a wavelength absorbed by a photosensitising compound.

The method may be for treating and/or preventing conditions caused by microorganisms in the oral cavity of a subject. The subject may be an animal or a human.

Similarly to the other method and/or use described above, the photosensitising compound may be comprised in the photosensitising composition.

The treatment and/or prevention may further comprise a step of waiting a predetermined period of time between steps (a) and (b) above. The step of waiting for a predetermined period of time between steps (a) and (b) may be from about 1 minute to 5 days. The waiting step may be from about 10 minutes to 3 days. The waiting step may be from 20 minutes to 1 day. The waiting step may be from 30 minutes to 5 hours.

The steps described above may be repeated as often as required, for example until the condition has reduced to a desired level or has been eliminated. The steps may be repeated after intervals of predetermined periods. The interval for the repeatability of the steps would be obvious to a person skilled in the art. For example, the steps may be repeated every few hours, every day, every 2 or 3 days.

According to another aspect the present invention provides a kit for treating and/or preventing conditions caused by microorganisms in a subject, the kit comprising the photosensitising composition according to any aspect of the present invention, disposed in at least one suitable container. The kit may be for treating and/or preventing conditions caused by microorganisms in the oral cavity of a subject. For example, the kit may be used for treating and/or preventing conditions caused by microorganisms, such as periodontal conditions and halitosis conditions. The conditions include, but are not limited to, gingivitis, periodontitis dental caries, root caries, root canal infection, apical periodontitis and the like. The kit may also be used for elimination of bacterial biofilm in any localised infection and/or managing bacteria deep within dental caries lesions. The subject may be an animal or a human.

The at least one photosensitising compound may be comprised in the photosensitising composition in the kit. Alternatively, the at least one photosensitizing compound may be disposed in a separate container or may be sold separately. The kit may further comprise instructions on the use of the composition. The kit may also comprise at least one light emitting device capable of emitting light at a wavelength absorbed by the at least one photosensitising compound. The photosensitizing compound may be an endogenous photosensitiser.

The present invention also provides a method of preparing the photosensitising composition described above. The method may comprise the step of: (a) preparing a mixture by mixing: at least one surfactant; at least one alcohol; and/or water, with the proviso that when the at least one surfactant is an alcohol, it is different from the at least one alcohol.

The alcohol and surfactant are any suitable alcohol and surfactant respectively, as described above. The at least one alcohol may be a monohydric alcohol, a dihydric alcohol and/or a trihydric alcohol, in particular, the alcohol is ethanol. In particular, the surfactant may be a trihydric alcohol or polyester. The trihydric alcohol may be glycerol. The polyester may be polyethylene glycol. Further, if the surfactant is an alcohol, the at least one alcohol is different from the surfactant.

According to a particular aspect, the photosensitizing composition may be prepared by mixing glycerol, ethanol and water. According to another particular aspect, the photosensitizing composition may be prepared by mixing polyethylene glycol, ethanol and water.

The method may comprise the step of preparing the mixture by mixing the at least one surfactant, the at least alcohol and water in a volume ratio in the range 10:5:85 to 40:30:30, with the proviso that when the at least one surfactant is an alcohol, it is different from the at least one alcohol. In particular, the ratio is in the range of 15:10:75 to 35:25:40. Even more in particular, the ratio is 30:20:50.

According to a particular aspect, the method comprises preparing a mixture of glycerol, ethanol and water, wherein the volume ratio of glycerol to ethanol to water in the mixture is in the range of 10:5:85 to 40:30:30. In particular, the ratio is in the range of 15:10:75 to 35:25:40. Even more in particular, the ratio is 30:20:50. The method may further comprise the step of adding at least one suitable photosensitising compound, as described above, to the mixture of step (a).

Having now generally described the invention, the same will be more readily understood through reference to the following examples which are provided by way of illustration, and are not intended to be limiting of the present invention.

### EXAMPLES

### Example 1

### Effect of PDT on Tooth Specimen

Tooth sections for this experiment were prepared and sterilised by autoclaving. These tooth specimens were then kept in 24 well plates. All Culture (AC) medium containing Enterococcus faecalis cells were added in each well at an optical density (OD) of 0.5. The plates were incubated under aerobic condition at 37° C. for 60 hours. The tooth sections were later removed from the micro well plates and was washed using sterile water to remove all planktonic bacterial ceils. Methylene blue solution (MB) (Sigma Aldrich, St. Louis, Mo.) was used for 10 minutes to photosensitise the E. faecalis biofilm on the tooth specimens. A low level laser light (660 nm from a diode laser source at 30 mW power) was used for light activated therapy in this experiment.

Group 1: This group consisted of tooth specimens that were not exposed to any treatment procedure. This group was also taken to be the positive control for the experiment.

Group 2: This group consisted of tooth specimens, to which a mixture of mineral oil, which is a refractive index matching liquid; and oxygen carrier, perfluorodecahydro naphthalene, was added and the specimens were photosensitised. The mixture comprised mineral oil and oxygen carrier in a volume ratio of 50:50.

Group 3: This group consisted of tooth specimens to which a mixture of glycerol, which is a refractive index matching liquid; and oxygen carrier, perfluorodecahydro naphthalene, was added and the specimens were photosensitised. The mixture comprised glycerol and oxygen carrier in a volume ratio of 50:50.

Group 4: This group consisted of tooth specimens to which a mixture of mineral oil, a refractive index matching liquid; dilute sodium hypochlorite, an oxidising agent (oxidant) that can pre-activate the photosensitiser; and oxygen carrier, perfluorodecahydro naphthalene, was added. The specimens were then photosensitised. The mixture comprised mineral oil, dilute sodium hypochlorite and oxygen carrier in a volume ratio of 46:45:10.

Group 5: This group consisted of tooth specimens to which a 500 μL of mixture of mineral oil, a refractive index matching liquid; and oxygen carrier, perfluorodecahydro naphthalene, was added. The specimens were then photosensitised. However, light illumination was carried out perpendicular to the root canal surface (wall).

Group 6: This group consisted of tooth specimens that were treated with 5.25% hypochlorite.

The specimens in Groups 2 to 5 were irradiated with light for 10 minutes. After light irradiation, the tooth specimens were split open and using a slow speed micromotor bur (1 mm in diameter) (DENTSPLY), dentinal shavings were removed for a depth of 1 mm from the root canal wall. The dentine shavings were mixed with 1 mL of AC medium (Sigma Aldrich, St. Louis, Mo.) and incubated aerobically at 37° C. After 1 hour, 10 μL of this medium was drawn and plated on previously prepared sterile AC agar plate. The bacterial count was determined from those plates after 18 hours of aerobic incubation.

The results, as provided in FIG. 1, showed that the specimen of group 2 and group 3 showed 80% and 90% reduction in the microbial count compared to the control of group 1. The specimen of groups 4 to 6 showed 100% reduction in the bacterial count.

Further, the teeth specimen treated with MB stained blue for ail treated groups, i.e. groups 2 to 5, except for group 4. The blue colour disappeared for ail the specimens in group 4. This showed that the treatment to the specimen of group 4 was the most effective among all the different groups as the colour of the teeth specimen was retained and that complete reduction in the bacterial cells was achieved. The specimen of group 5 also showed a 100% reduction in bacterial cell number. The specimens of groups 2 and 5 were exposed to the same conditions, but the specimens of group 2 only showed an 80% reduction as compared to a 100% for the specimens of group 5. This shows the importance of the angle of irradiation, wherein when the irradiation is perpendicular to the root canal, the effect is greater.

From the above, it can be seen that PDT is effective in eliminating bacteria in dentinal tubules.

### Example 2

E. faecalis was grown as biofilm in a 96-well collagen coated plate. Different growth media were used as follows:

a) All Culture (AC);

b) AC and Glycerol, in a volume ratio of 50:50;

c) serum; and

d) serum and glycerol, in a volume ratio of 50:50.

The plate was incubated at 37° C. for 12 hours under aerobic conditions. After the incubation period, the medium from the wells was removed and the wells were washed with 1×PBS to remove any planktonic cells. The wells were then filled with methylene blue solution (100 μg/mL) for 15 minutes for photosensitisation. Subsequently, the wells were filled either with glycerol, oxygen carrier or a mixture of glycerol and oxygen carrier. The oxygen carrier used was perfluorodecahydro naphthalene.

A control group was also included, in which the wells which served as control were not given any treatment, such as photosensitisation. A further group included wells which had the chemicals added to the wells, but were not irradiated with light. This group served to indicate the effect of light activation/irradiation on the bacteria.

Low level light (660 nm Laser from a diode laser of at 30 mW power) was used in this study for 5 minutes. Subsequent to the irradiation, the wells were filled with AC medium and incubated for 3 hours. 10 μL of this solution was taken and plated on AC agar. The number of cells present in each test group was counted after 12 hours.

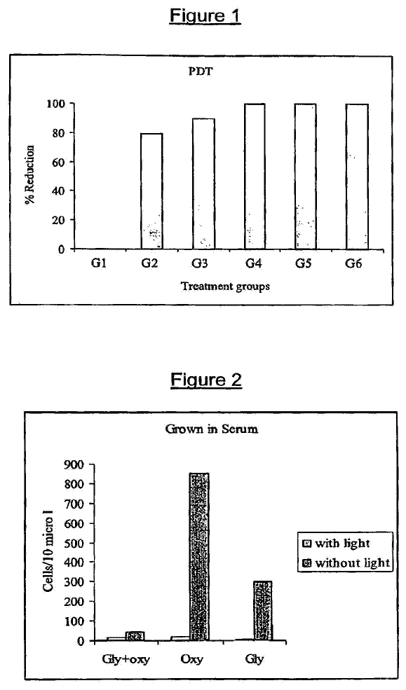

FIGS. 2 and 3 show that the proposed method is effective in eliminating E. faecalis growing as a biofilm in root canal dentine. However, it was also observed from FIG. 2 that the effectiveness of the treatment on the bacterial ceils depended on the combination of chemicals used. This shows that this treatment approach is effective even in presence of serum.

The efficacy of the irradiation was also significantly evident from the results shown in FIG. 2, especially when photosensitisation was coupled with oxygen carrier and glycerol, a refractive index matching liquid.

### Example 3

### Materials and Method

Methylene blue (MB) (Sigma Aldrich, St Louis, Mo.), a phenothiazine dye which was purified by ethanol extraction was used in this experiment. All the chemicals used in the experiment were of analytical grade. Four different formulations of media were tested for their efficiency to enhance the photosensitisation effect: (1) Water; (2) 70% Glycerol; (3) 70% Poly Ethylene Glycol (PEG) (MW200); and (4) a mixture of Glycerol:Ethanol:Water (volume ratio of 30:20:50) (herein referred to as “Mix”). The light source used was diode laser having wavelength of 664 nm (LDCU/6130, Power Technology Inc, Little Rock, Ark., USA). All chemicals were purchased from Sigma Aldrich, St Louis.

The photophysical characterisations of MB in different media were carried out by analysing the absorption and emission spectra. Further, the suitability of the formulations in penetrating into the dentinal tubules, bacterial cells and finally the elimination of biofilm bacteria under both in vitro and ex vivo conditions were also analysed.

(a) Spectroscopic Studies

Absorption spectra of methylene blue in different formulations were determined using a UV/VIS Spectrophotometer (Shimadzu 1100, Japan). The ratio of absorbance at 664 to that at 612 nm was calculated, and plotted as an index of monomer to dimmer in different media. The fluorescence intensity of increasing concentration of MB (6.25, 12.6, 25, 50, and 100 μM) was measured using a fluorimeter (Perkin Elmer) at room temperature. In particular, the conditions were as follows: excitation wavelength: 650 nm; emission wavelength: 680 nm; EX slit: 2.5 nm; emission slit: 20 nm; and Em filter 515 nm).

(b) Photosensitiser Penetration into Dentinal Tubules

12 human teeth (both single rooted and multiple rooted) with no history of caries and/or defects were selected for the experiment. The crown portion of all samples was cut at the level of cemento enamel junction, to get an approximate length of 8-10 mm. The root canal was cleaned using root canal irrigants (NaOCl and EDTA 1% and 100 mM respectively). These specimens were washed and kept upright in 96-well plates containing 2% agar. As the agar solidifies, the tooth specimen sits in a socket formed of agar. 100 mM Methylene blue (MB), the photosensitiser, in different media was introduced into the root canals by using a syringe and was kept at 37° C. for 30 minutes. The excess MB remaining in the root canal was blotted by using paper points. Thin cross-sections of these specimens were prepared by using a Micro Slice Machine (Metal Research, England). The cross-sections were scanned using HP Scanjet 3970 (Hewlett-Packard, Calif., USA). The extent of diffusion of MB was measured using an image tool software. MB diffusion into dentinal tubules was measured at 3 regions: cervical (first three sections of a single root), middle (three sections from middle portion of the specimen) and apical (last three sections of a single root). The percentage MB penetration into dentinal tubules was calculated by dividing the distance diffused by MB in front from the lumen of root canal to the total length from the lumen to outer surface.

(c) Photosensitizer Uptake by Bacterial Cells

Two endodontic pathogens. Enterococcus faecalis (ATCC 29212), and Actinobacillus antinomycetemcomitans (ATCC 33384) were used in this experiment. The former pathogen is a gram positive bacteria and the latter is a gram negative bacteria. Bacterial cells were grown aerobically in Brain Heart Infusion (BHI) broth. Cells in the stationary phase of growth were harvested by centrifugation and were washed with Of water. The optical density of the culture was adjusted to 1 at 600 nm and that corresponded to 10⁸ cells/mL. Cells harvested from 1 mL of the above suspension were added to 100 μM of MB in different formulations and incubated at 37° C. for 30 minutes. The cells were harvested and washed twice with DI water. 1 mL of 2% SDS was added to the harvested cells and kept at 37° C. for 16 hours. The optical density of the supernatant solution after centrifugation was recorded at 663 nm.

(d) PDT on Biofilms Produced in Multiwell Plates

Two-day old biofilms of E. faecalis and A. actinomycetemcomitans were produced in multiwell plates in AC and BHI media respectively. After the incubation time, the media was removed from each of the wells. The entire process of PDT was conducted in 2 steps: in the first step, the biofilm bacteria was sensitised with MB (sensitising media) and in the second step, irradiation was conducted by adding an oxygen carrier (irradiating media). The oxygen carrier can ensure proper supply of oxygen and hence enhance the photodynamic action. In addition, oxygen carriers such as Perfluorodecahydro naphthalene increases the lifecycle of a singlet oxygen. The wells were first filled with 1 mM of MB in each of the four test formulations and subsequently photosensitised for 10 minutes. In the second phase, the wells were replaced with oxygen carrier solution of Perfluorodecahydro naphthalene, and irradiated for 10 minutes (63.694 J/cm²) with a diode laser emitting light at a wavelength of 664 nm (LDCU/6130, Power Technology Inc, Little Rock, Ark., USA) under constant shaking on a plate shaker. After treatment, the wells were replaced with fresh growth media and vigorously shaken. Biofilm on the wails of wells were mechanically disrupted to get the bacteria into the media. Appropriate dilutions of the growth media from wells were made and plated to enumerate the surviving bacterial cells.

(e) Ex Vivo Experiment

(i) Preparation of Tooth Specimens

Seventy two single rooted non-caries teeth were selected for the experiment. Tooth sections were prepared by removing crown at the level of Cemento Enamel junction and sectioning the apical third of the root to obtain a standard length of 8 mm. The root canal region of these specimens were prepared using endodontic files together with 1% hypochlorite solution and 100 mM EDTA solution. Half of the tooth specimens were incubated with E. faecalis and the other half were incubated with A. actinomycetemcomitans in All Culture media and BHI respectively for 4 days. Biofilm formation in dentine takes more time than in multiwell plates. The growth medium was replaced every 2 days with fresh medium.

(ii) Photodynamic Treatment (PDT)

Tooth specimens with bacterial biofilm in root canals were removed from the culture and were positioned with the coronal root canal orifice facing upward by embedding them in 2% agar in single wells of a 24 well-plate. This positioning of the specimens simulated in vivo conditions, where the root portion of a tooth is housed in the alveolar bone socket. Initially, the culture medium remaining in the root canal was removed by using a micropipette, PDT was performed in two steps, as with the in vitro experiment, explained above. Root canal space was filled with either the sensitising media or the irradiation media according to phase of the treatment.

100 μL of photosensitiser formulation (sensitising media) was added to the root canal and photosensitisation was conducted for 30 minutes at 37° C. The photosensitiser formulation was removed by pipetting out the excess amount, and the root canal was then filled with irradiation media. Irradiation was performed using a diode laser of wavelength 684 nm by using an optical fibre. The terminus of the optical fibre was placed at the coronal end of the root canal and irradiation was carried out for 20 minutes (114.64 J/cm²). Subsequently, the irradiation media was removed, grooves made on the proximal side of the tooth specimen, and using a chisel and hammer, the specimens were split open. Dentine shavings were obtained from the root canal region (2 mm away from the opening) using a round burr of 1.5 mm diameter, held straight and perpendicular to the roof canal surface. Dentine shavings were added to fresh AC media and appropriate dilutions were made. 10 μL was transferred to AC plate and spread plated. Bacterial counting was carried out after 24 hours of incubation at 37° C.

### Results

(a) Spectroscopic Studies

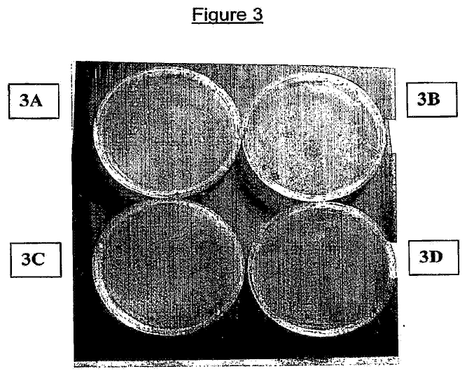

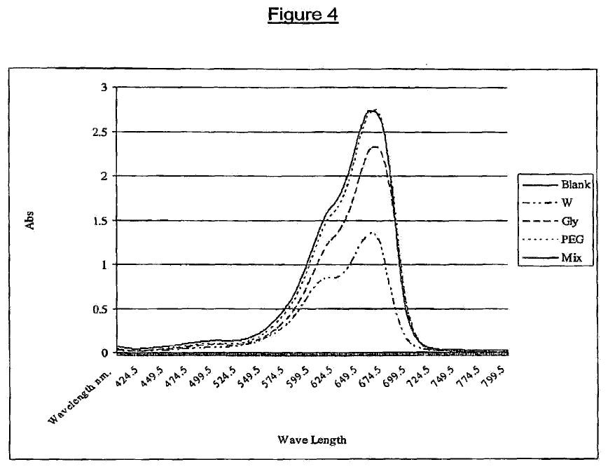

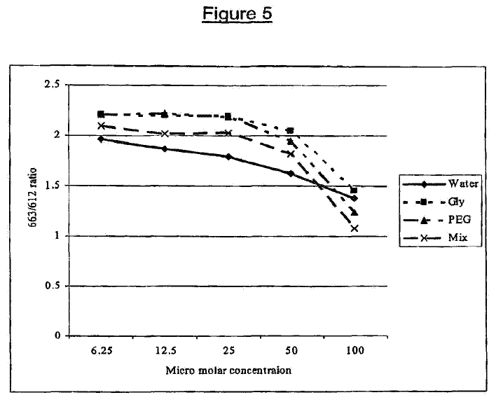

The results from the absorption spectra of methylene blue (MB) in different formulations showed an increase in the absorption when dissolved in PEG and Mix compared to other formulations of media, as shown in FIG. 4. The spectrum shows 2 bands peaking; at 612 nm, corresponding to the dimmer; and a peak maxima at 664 nm, corresponding to the monomer. A slight shift of absorption maxima from 664 nm to 667 nm was observed when MB was dissolved in media other than water. However, as the molar concentration of MB increased (i.e. from 6.25 μM to 100 μM), the absorption maxima shifted to the peak corresponding to the dimmers, indicating the aggregation of photosensitiser molecules, as shown in FIG. 5. The monomer to dimmer ratio is lower for MB in water compared to different formulations at lower concentrations. Therefore, at lower concentrations, the aggregation of photosensitiser molecules was lessor in formulations other than water, as can be seen from the monomer to dimmer ratio in FIG. 5, However, as the concentration of methylene blue increased, the 612 nm peak became more predominant compared to the 664 nm peak.

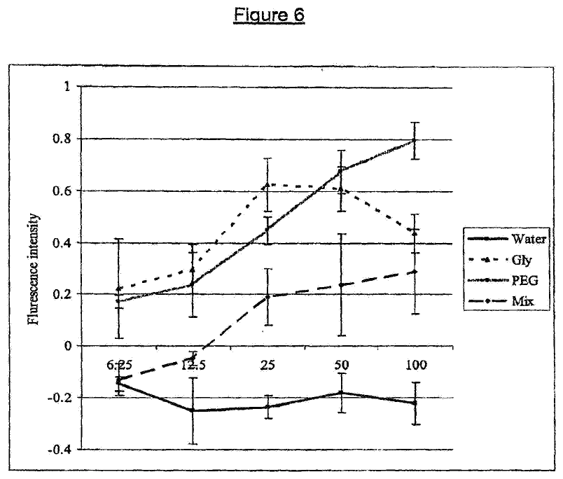

FIG. 6 shows the fluorescence intensity of MB in different formulations. MB in water showed no fluorescence emission. The formulation of PEG showed an approximate linear increase in the fluorescent intensity as the concentration of MB increased. The maximum emission was seen at 680 nm. There was only a marginal change in the emission maxima of MB in different formulations. The fluorescence intensity was found to increase in formulations of PEG and Mix as the concentration of MB increased.

(b) Photosensitiser Penetration in Dentinal Tubules

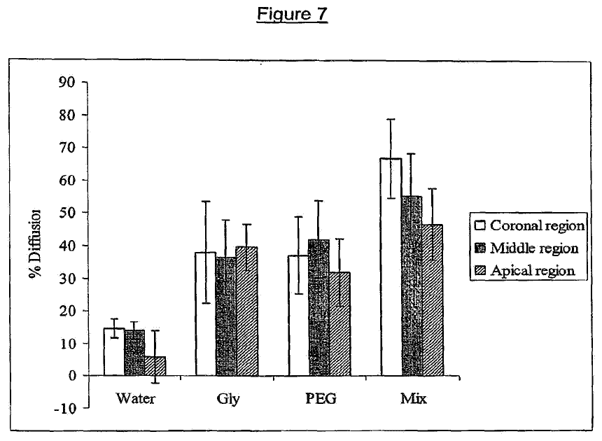

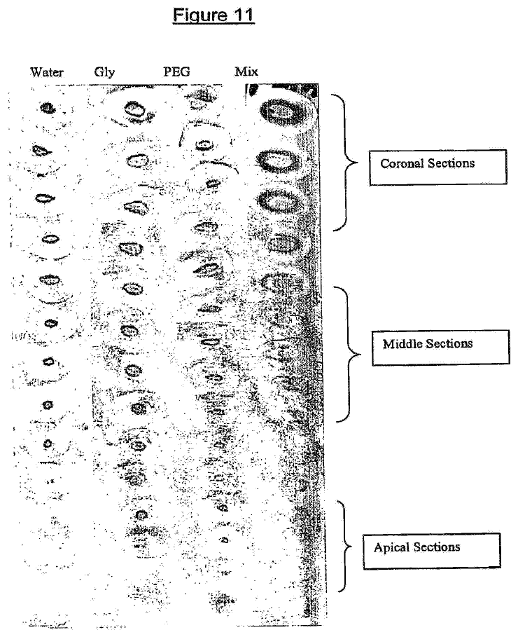

The percentage penetration of MB, the photosensitiser, across the dentinal tubules is shown in FIG. 7. The penetration of MB in dentinal tubules was studied from three regions of interest in the tooth: coronal; middle; and apical regions. MB based on Mix formulation showed maximum penetration in all the regions of interest under the test conditions described above. MB in water showed the least penetration across the dentinal tubules. The results are further shown in FIG. 11.

(c) Photosensitiser Uptake by Bacterial Cells

The accumulation of MB by both gram positive and gram negative bacteria was tested as described above. FIG. 8 shows the percentage of photosensitiser uptake by E. faecalis and A. actinomycetemcomitans. Gram positive bacteria was found to have a higher MB uptake than gram negative. Maximum MB uptake in both bacteria, as indicated by the absorbance measurement, was found in the formulation of water. Among the other formulations, Mix had a higher MB uptake by bacterial cells.

(d) PCT on Biofilm Produced in Multiwell Plates

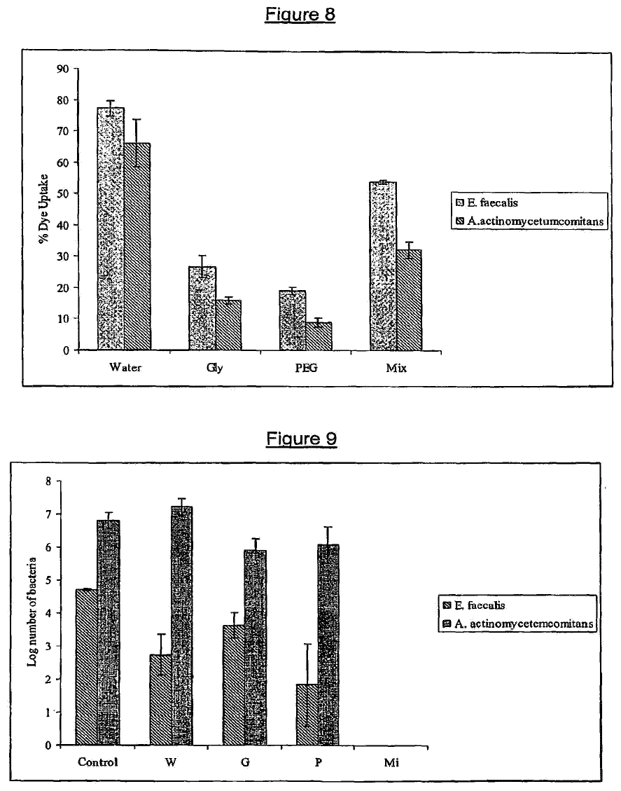

FIG. 9 shows the log number of E. faecalis remaining after PDT treatment. 100% bacterial elimination was seen in formulation based on Mix, followed by the formulation of PEG. In the biofilm of A. actinomycetemcomitans. Mix showed maximum bacterial elimination (100%). However, the other formulations were not as effective as the Mix. The results of FIG. 9 also show that it is the combination of photosensitising and irradiation that produces the desired effect of bacterial killing. Irradiation or photosensitising alone does not have an effect on bacterial elimination.

(e) Ex Vivo Experiment

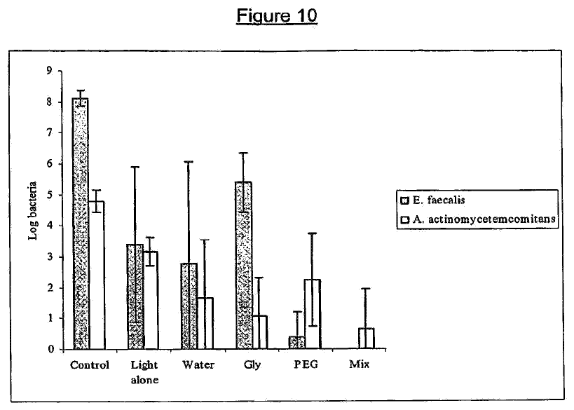

The result of PDT on biofilm developed in root canal showed varying percentages of bacterial elimination according to the formulations used (FIG. 10. The pattern of bacterial elimination by formulations was similar to those seen in biofilm developed in multiwell plates. In the case of E. faecalis, the formulation of Mix showed 100% bacterial elimination. Significant bacterial reduction was also observed in formulation based on PEG.

PDT performed in root canals with A. actinomycetemcomitans biofilm also showed a similar trend. The formulation of Mix showed 86.5% reduction in bacterial count compared to the control group. However, generally, the susceptibility of A. actinomycetemcomitans to the bactericidal action of PDT was lower compared to that of E. faecalis.

### Discussion

The above results show that light absorption property of MB differed in different formulations. The absorption capacity of MB increased two fold when dispersed in PEG and Mix compared to MB in water. The monomer to dimmer ratio as shown in FIG. 5 indicates the capacity of formulations such as PEG and Mix to prevent aggregation of MB at lower concentration. It was observed that at higher concentration, MB aggregation was more in the formulations of PEG and Mix, compared to that of water. The fluorescence intensity was also found to steadily increase with an increase in concentration when MB was dispersed in Mix and PEG. The spectroscopic studies suggest that the photophysical characteristics of MB dispersed in Mix and PEG are suitable for PDT.

The proper diffusion of photosensitiser to anatomical complexities and dentinal tubules, which can potentially shelter bacteria from treatment, is a prerequisite of a photosensitising composition for PDT targeted to eliminate bacteria from endodontic milieu. Agents with reduced surface tension could be the one that can help maximum penetration across dentinal tubules. Glycerol, due to its high density could be a possible candidate to obtain maximum penetration into root canal since the flow will be driven by gravity. However, glycerol based formulations may not be effective in treating maxillary teeth infections. An optimised formulation should be able to target both mandibular and maxillary teeth. Results from the MB diffusion into the dentine showed that the formulation of Mix facilitated maximum penetration. Since the penetration of any liquid into the dentinal tubules is influenced by the taper of the root canal and diameter of dentinal tubules, three regions of interest were considered at (1) cervical, (2) male and (3) apical regions of the root to assess the depth of penetration. The MB penetration study indicated a penetration depth higher than 1.5 mm in the coronal region. Hence, using the formulation of Mix, it would be possible even to target bacterial cells invading dentinal tubules. In addition, current intracanal medicaments used as disinfectants are found to be neutralized by dentine (Haapasalo H K et al, 2000) end also the antimicrobial properly of current disinfectants reduced deeper in the dentine (Heling I Chandler N P 1998).

A formulation will only be effective if it helps the photosensitiser uptake by bacterial cells or its association with bacterial membrane. Close vicinity of the photosensitiser to the target is necessary since the singlet oxygen and other reactive oxygen species produced during PDT will diffuse only a maximum distance of 20 nm (Rokitskaya T I et al, 2000; Valenzeno D P, 1987). Further, the lifespan of these radical species in aqueous medium is lesser than in lipid membranes. Since infection of root canal system (endodontic sites) represents multi-bacterial consortia, the formulation should facilitate photosensitiser uptake by both gram negative and gram positive bacteria. Although gram positive bacteria accumulates photosensitisers, the presence of the outer membrane in gram negative bacteria makes them less permeable to photosensitisers. Photosensitising compositions that induce the outer membrane permeability of gram negative bacteria and hence susceptibility, have been successfully tried in many therapeutic approaches. Water based MB formulation showed maximum uptake of MB by both gram negative and gram positive organisms (A. actinomycetemcomitans and E. faecalis respectively). After the formulation of water, Mix was also found to facilitate MB accumulation in the cells. Although the absorbance-based experiments showed maximum MB intake in the water formulation, fluorescence intensity was found to be minimum in water. The lowered fluorescence intensity could be due to the self-quenching because of the increased concentration of MB in the cells. PDT has been suggested as a potential treatment, regime to fight antibiotic resistant bacterial infections involving biofilm (Gander S, 1996). The physiochemical features of biofilm with altered genotypic and phenotypic characteristics of indwellers protect them from routine antimicrobial agents. However, since the killing is mediated by oxygen free radicals in PDT, the chance of bacteria resisting and acquiring resistance upon long term usage could be minimum. Since penetration of photosensitiser is hindered by the structural and chemical nature of biofilm, appropriate formulations should be screened. Hence the effectiveness of different formulations in eliminating biofilm bacteria was tested under in vitro and ex vivo conditions, irradiation alone or photosensitising alone had no effect on the viability status of bacteria as revealed by CFU counting. 100% bacterial elimination was seen in E. faecalis biofilm when irradiated after sensitising with Mix. E. faecalis is recognized as one of the human pathogens with high resistance to many antimicrobial agents. In addition, recent emergence of antibiotic resistant strains of this bacterium, pose a great challenge to medical community. Although the presence of E. faecalis in primary root canal infection is comparatively lesser, it has been isolated in high proportion or as single isolate in failed root canal therapies (secondary infection). Many environmental factors existing in root canal after treatment, as well as physiological properties of E. faecalis, has been proposed to explain the increased prevalence of this bacterium in secondary infection. Conventional root canal treatment has limitations in circumventing factors such as bacterial penetration into dentinal tubules, its ability to resist chemicals, and production of biofilm on root canal and associated regions. Since PDT using the formulation of Mix showed 100% elimination of bacteria from biofilm produced on inanimate material and even dentine, its use as an endodontic treatment regime is warranted.

When compared to E. faecalis biofilm, PDT was found to be less effective in eliminating A. actinomycetemcomitans biofilm. However, Mix showed the best results among the other formulations (100% and 86.5% respectively with Mix) in eliminating A. actinomycetemcomitans. As stated above, the elimination was harder for A. actinomycetemcomitans as it is a gram negative bacteria. However, increasing the dose of irradiation may eliminate even gram negative bacteria.

### Example 3

The experiment described below was performed to assess the cytotoxic effect of PDT in combination with two photosensitisers; Indocyanine Green (ICG) and Methylene Blue (MB) in different formulations, and this was compared to sodium hypochlorite. Sodium hypochlorite is traditionally used to disinfect root canals.

(a) Materials and Method

### (I) Experiment 1—Indocyanine Green

Fibroblast L 929 was used in this experiment Approximately 3.2×10⁵ cells were seeded in 4 cm diameter culture dishes in DMEM media supplemented with 10% FCS. Cells were grown by incubating at 37° C., in a carbon dioxide incubator for 24 hours. After the incubation period, 100 μL of ICG (1 mM) was added (the final concentration of ICG was 100 μM). 100 μL of oxygen carrier, perfluorodecahydro naphthalene.] was also added and irradiation was performed for 20 minutes (without shaking). The cells were left in the media for 24 hours under incubation. Later, the supernatant media was removed without disturbing the cell line. The cell layer was washed with 1 mL PBS twice. MTT (0.125 mg % in DMEM), as an indicator of the viable status of cells in the media was added to the Petri dish and incubated at 37° C., for 4 hours. The excess of dye was washed with PBS (2 mL). MTT formed in the Petri dish was extracted and added to 1 mL of DMSO. The optical density at 570 nm was measured. Sodium hypochlorite (100 μL of 5.25%) was used as a control in this experiment. All chemicals were purchased from Sigma Aldrich, St. Louis.

### (II) Experiment 2—Methylene Blue

Fibroblast L 929 was used in this experiment. Approximately 3.2×10⁵ cells were seeded in 4 cm diameter culture dishes. Cells were grown by incubating at 37° C., in a carbon dioxide incubator for 24 hours. After the incubation period, 100 μL of methylene blue (MB) (1 mM) was added to the culture dishes until the final concentration of MB was 100 μM. 100 μL of oxygen earner, perfluorodecahydro naphthalene, was also added and irradiation was performed for 20 minutes (without shaking). The cells were left in the media for 24 hours under incubation. Later, the supernatant media was removed without disturbing the cell line. The cell layer was washed with 1 ml PBS twice. As in experiment 1 above, MTT was added in the Petri dish and incubated at 37° C. for 4 hours. The excess dye was washed with PBS (2 mL). MTT formed in the Petri dish was extracted to 1 mL of DMSO. The optical density at 570 nm was measured. Sodium hypochlorite (100 μL of 5.25%) was used as a control in this study.

(b) Results

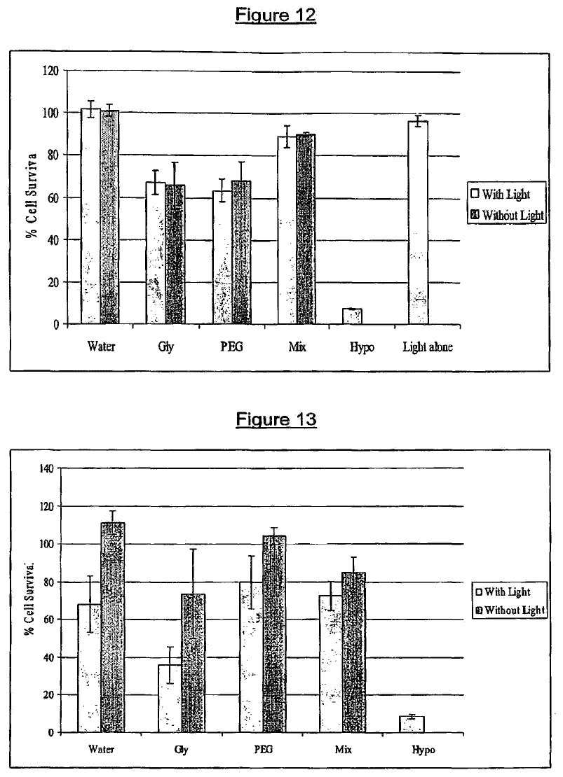

The graphs represent percentage cells surviving in different formulations after PDT using ICG (FIG. 12) and MB (FIG. 13). It was seen that the cytotoxicity was lessor for PDT when ICG was used as compared to using MB. In particular, PDT using ICG in water showed the least cytotoxicity. Comparing the effect of ICG and MB on Mix, ICG showed lesser cytotoxicity. Therefore, MB would be a better photosensitising compound. Further, sodium hypochlorite showed highest the cytotoxicity of all the formulations tested. The results show that conventional chemicals, such as sodium hypochlorite, used in treatment provide drastic killing of normal fibroblast cells, but Mix is effective against bacteria and suitable for fibroblasts cells.

However, as mentioned above, although sodium hypochlorite showed the highest cytotoxicity, combining the results from the other examples, it can be concluded that Mix is a more appropriate photosensitising composition compared to sodium hypochlorite.

### REFERENCES

Berkiten M, Okar I, Berkiten R. In vitro study of the penetration of Streptococcus sanguis and Prevotella intermedia strains into human dentinal tubules. J Endod. 2000 April; 26(4):236-9

Costerton, J, W., Lewandowski, Z., DeBeer D., Caldwell, D., Korber, D., and James, G. 1994. Biofilms, the customized microniche. J Bacterial 176:2137-2142

De Moor R J, Hommez G M, De Boever J G, Delme K I, Martens G E. Periapical health related to the quality of root canal treatment in a Belgian population. Int Endod J. 2000; 33(2):113-20

Detty M R, Gibson S L, Wagner S J, J Med Chem, Current chemical and preclinical photosensitizers for use in photodynamic therapy, 2004, 47(16):3897-915

Dougherty W J, Bae K S, Watkins B J, Baumgartner J C, Black pigmented bacteria in coronal and apical segments of infected root canals, Journal of Endodontics, 1996, 24(S):35S-8

Dugas N N, Lawrence H P, Teplitsky P E, Pharoah M J, Friedman S. Periapical health and treatment quality assessment of root-filled teeth in two Canadian populations. Int Endod J. 2003 March; 36(3):181-92

Eckerbom M, Andersson J E. Magnusson T. A longitudinal study of changes in frequency and technical standard of endodontic treatment in a Swedish population. Endod Dent Traumatol. 1989 February; 5(1):27-31

Economou-Stamatelopoulou C, Roussopoulos G P, Prouskas J C, Apostoiopoulos M. OPHTHALMOLOGICA 217 (6): 426-430 November-December 2003

Eriksen H M. Epidemiology of apical periodontitis. In: D Ørstavik, T R Pitt Ford, editors. Essential Endodontology, Prevention and treatment of apical periodontitis. London: Blackwell Science; 1998, p. 179-91

Figdor D, Apical periodontitis: Editorial, Oral Surg Oral Med Oral Pathol Oral Radiol Endod. 1996

Figdor, D. 2002. Apical periodontitis: A very prevalent problem. Oral surgery Oral medicine Oral pathology 94:851-652

Gander, S. 1996, Bacterial biofilms: Resistance to antimicrobial agents. Journal Of Antimicrobial Chemotherapy 37:1047-1050

Haapasalo, H. K., E. K. Siren, T. M. T. Waltimo, D. Orstavik, and M, P. P. Haapasalo. 2000. Inactivation of local root canal medicaments by dentine: an in vitro study. International Endodontic Journal 33:126-131

Haapasalo, M., Udnes, T., and Endal, U. 2006. Persistent, recurrent, and acquired infection of the root canal system post-treatment. Endodontic topics 6:29-56

Hamblin, M. R., and T. Hasan. 2004. Photodynamic therapy: a new antimicrobial approach to infectious disease? Photochemical & Photobiological Sciences 3:436-450

Hancock H H, Sigurdsson A, Trope M, Moiseiwitsch J. et al. Bacteria isolated after unsuccessful endodontic treatment in a North American population. Oral Surg Oral Med Oral Pathol Oral Radiol Endod. 2001 May; 91(5): 579-86

Heling, I., and N. P. Chandler, 1998 Antimicrobial effect of irrigant combinations within dentinal tubules, International Endodontic Journal 31:8-14

J D Whitney, Heart and Lung, 1989, 18, page 466, General information about high pressure oxygen in wound healing

Jori G and S B Brown, Photosensitized inactivation of microorganisms. Photochemical & Photobiological Sciences, 2004, 3(5):403-405

Love R M. Regional variation in root dentinal tubule infection by Streptococcus gordonii. J Endod. 1996 June; 22(6):290-3

Lupi-Pegurier L, Bertrand M F, Muller-Bolla M, Rocca J P, Sofia M. Periapical status, prevalence and quality of endodontic treatment in an adult French population. Int Ended J. 2002 August; 35(8):690-7

Mah T F C, O'Toole G A (2001), Mechanisms of biofilm resistance to antimicrobial agents. Trends Microbiol 9:34-39

Marsh, P D, 1994 Microbial ecology of dental plaque and its significance in health and disease. Adv. Dent. Res. 1994, 8, 263-271

Michael R W, Anthony 8, Smith W (2001). Dormancy and persistence in chronic infection: role of the general stress response in resistance to chemotherapy. J Antimicrob Chemother 48:141-142

Nair, P. N. R. 2004. Pathogenesis Of Apical Periodontitis and the Cause of Endodontic Failures. Critical Reviews in Oral Biology & Medicine 15:348-381

Noiri Y, Ehara A, Kawahara T, Takemura N, Ebisu N S (2002). Participation of bacterial biofilms in refractory and chronic periapical periodontitis. J Endod 28:679-683

O'Neill J F, C K Hope and M Wilson, Oral Bacteria in multi-species biofilms can be filled by red light in the presence of toluidine blue. Lasers in Surgery and Medicine, 2002, 31(2):85-90

Ochsner M, 1997, Photophysical and photobiological processes in the photodynamic therapy of tumours. J. Photochem Photobiol B:Biol.; 39:1-18

Parsek, M. R,, and Singh P. K. 2003. Bacterial biofilms: Emerging link to disease pathogenesis. Annual review of microbiology 57:677-701

Perez F, Calas P, de Falguerolles A, Maurette A. Migration of a Streptococcus sanguis strain through the root dentinal tubules. J Endod. 1993a June; 19 (6):297-301

Perez F, Rochd T, Lodter J P, Galas P, Michel G. In vitro study of the penetration of three bacterial strains into root dentine. Oral Surg Oral Mod Oral Pathol 1993 July; 76(1):97-103

Pervaiz S, Reactive oxygen-dependent production of novel photochemotherapeutic agents, Faseb Journal, 2001, 15(3):612-617

Rokitskaya, T. I., Block, M., Antonenko, Y. N., Kotova E. A., and Pohl P. 2000. Photosensitizer Binding to Lipid Bilayers as a Precondition for the Photoinactivation of Membrane Channels. Biophysical Journal 78:2572-2680

Soncin M, Fabris C, Busetti A, Dei D, Nistri D, Roncucci G, Jori G, Approaches to selectivity in the Zn(II)-phthalocyaninephotosensitized inactivation of wild-type and antibiotic-resistant Staphylococcus aureus, Photochem. Photobiol, Sci., 2002, 1:818-819

Soukos N S et al, Targeted antimicrobial photochemotherapy, Antimicrobial Agents and Chemotherapy, 1998, 42(10):2595-2601

Stewart P S (1996). Theoretical aspects of antibiotic diffusion into microbial biofilms. Antimicrob Agents Chemother 40:2517-2522

Stewart P S, Costerton J W (2001). Antibiotic resistance of bacteria in biofilms. Lancet 358:135-138

Sundqvist G. Ecology of the root canal flora. J Endod. 1992 September; 18 (9):427-30

Tronstad, L., and Sunde, T. P. 2003. The evolving new understanding of endodontic infections. Endodontic topics 6:57-77

Valenzeno, D. P. 1967. Photomodification of biological membranes with emphasis on singlet oxygen mechanisms. Photochem. Photobiol 46:147-160

Wainwright, M. 1998. Photodynamic antimicrobial chemotherapy (PACT). Journal Of Antimicrobial Chemotherapy 42:13-28

Wainwright, M., and Crossley. K. B. 2004. Photosensitizing agents—circumventing resistance and breaking down biofilms: A review, International biodeterioration and biodegradation 53:119-126

Yoshida H et al., Applied Optics, 36(16), pp 3739-3744, 1997

1. A photosensitising solution for use in photodynamic elimination of a microorganism in dentinal tubule of a root canal, the photosensitising solution comprising at least one photosensitising compound dissolved in an alcohol carrier solution,
wherein the at least one photosensitising compound being selected from the group consisting of: toluidine blue, methylene blue, arianor steel blue, tryptan blue, crystal violet, azure blue cert, azure B chloride, azure II, azure A chloride, azure B tetrafluoroborate, thionin, azure A eosinate, azure B eosinate, azure mix sicc., azure II eosinate, haematoporphyrin HCl, haematoporphyrin ester, aluminium disulphonated phthalocyanine, chlorins, photoactive fullerenes (e.g. C16-b), aminolevulinic acid (ALA), bacteriochlorins, phthalocyanines, pheophorbides, purpurins, naphthalocyanines, indocyanine green, and mixtures thereof;
wherein the alcohol solution comprises polyethylene glycol and/or ethanol.

2. The photosensitising solution according to claim 1, wherein the at least one photosensitising compound is methylene blue or arianor steel blue.

3. The photosensitising solution according to claim 1, wherein the alcohol carrier solution comprises polyethylene glycol and ethanol.

4. The photosensitising solution according to claim 1 wherein the at least one photosensitising compound has a concentration of about 100 micromolar.

5. The photosensitising solution according to claim 1, wherein the polyethylene glycol, ethanol and water of the carrier solution are combined in a ratio of 1.3:1:1 to 2:1:17.

6. The photosensitising solution according to claim 1, wherein the polyethylene glycol, ethanol and water of the carrier solution are combined in a ratio of about 30:20:10.
